(19) United States
(12) Patent Application Publication
(10) Pub. No.: US 2008/0286809 A1
(43) Pub. Date: Nov. 20, 2008

(54) Methods

(75) Inventors: Dario Renato Alessi, Scotland (GB); Alberto Clemente Vitari, Scotland (GB)

Correspondence Address: SHERIDAN ROSS PC, 1560 BROADWAY, SUITE 1200, DENVER, CO, 80202, US

(73) Assignee: MEDICAL RESEARCH COUNCIL, London (GB)

(21) Appl. No.: 11/995,893

(22) PCT Filed: Jul. 14, 2006
(86) PCT No.: PCT/GB2006/002634

§ 371 Date: Jun. 27, 2008

(30) Foreign Application Priority Data
Jul. 16, 2005 (GB) 0514661.8

(51) Int. Cl. G01N 33/566; C12N 9/12; C12N 15/54

(52) U.S. Cl. 435/7.8; 435/194; 536/23.2

(57) ABSTRACT

A method for identifying a compound expected to be useful in modulating a WNK (With No Lysine Kinase) isoform protein kinase activity, the method comprising the steps of (1) determining whether a test compound modulates the protein kinase activity of a WNK isoform polypeptide on the substrate SPAK (STE20/SPS1-related Proline-Alanine-rich Kinase) or OSR1 (Oxidative Stress Response kinase-1) and (2) selecting a compound which modulates the said WNK isoform polypeptide protein kinase activity. Such a compound may be useful as an antihypertensive agent or for the treatment of, for example, Gordon's syndrome.

The present invention relates to assays and substrates relating to WNK1 and WNK4 protein kinases.

The WNK family of protein kinases comprise 4 members (WNK1, WNK2, WNK3 and WNK4) and were originally identified as distinctive serine-threonine protein kinases that lack a conserved Lys residue normally found in subdomain II of the catalytic domain [1, 2]. Subsequent studies identified mutations in the genes encoding WNK1 and WNK4 in families with an inherited hypertension and hyperkalemia (elevated serum K⁺) disorder, called pseudohypoaldosteronism type II (PHAII, also known as Gordon's syndrome) [3]. WNK isoforms are large protein kinases (WNK1-2382 residues, WNK4-1243 residues), in which the catalytic domain is located at the N-terminus (residues 221 to 479 for WNK1 and 174 to 432 for WNK4). Apart from two putative coiled-coil domains, the remainder of the WNK polypeptides possess no obvious structural features. Mutations in the WNK1 gene found in PHAII subjects are deletions in intron-1, which reportedly elevate the expression of the WNK1 protein, indicating that hypertension could result from increased expression of WNK1 [3]. Consistent with this notion, mice lacking one allele of WNK1, and which express reduced levels of WNK1 protein, have lower blood pressure [4]. The WNK1 knockout is an embryonic lethal, indicating that WNK1 is also required for normal development [4]. Thus far, the mutations in the WNK4 gene found in PHAII subjects lie distal to both of the putative coiled-coil domains [3,5].

Most functional studies on WNK1 and WNK4 have focused on the overexpression of these enzymes and monitoring of the effects that this has on co-expressed ion channels or cotransporters in Xenopus oocytes or para-cellular ion flux through tight junctions in epithelial cells [6-8]. For example, overexpression of WNK4 markedly inhibited Na⁺ flux mediated by the sodium chloride cotransporter, by reducing its level at the plasma membrane [9, 10]. Overexpression of WNK4, in MDCK epithelial cells, increased para-cellular ion flux through tight junctions, an effect that was postulated to result from phosphorylation of claudins, a family of transmembrane tight junction proteins [11]. It was recently reported that WNK1 interacted with and phosphorylated synaptotagmin-2, a Ca²⁺ sensor, which regulates endocytosis and exocytosis and may affect trafficking of ion channels to the plasma membrane [12].

We have identified two related protein kinases of the STE20 family that are phosphorylated and activated by WNK isoforms.

A first aspect of the invention provides a method for identifying a compound expected to be useful in modulating a WNK (With No Lysine Kinase) isoform protein kinase activity, the method comprising the steps of (1) determining whether a test compound modulates the protein kinase activity of a WNK isoform polypeptide on a substrate SPAK (STE20/SPS1-related Proline-Alanine-rich Kinase) polypeptide or OSR1 (Oxidative Stress Response kinase-1) polypeptide and (2) selecting a compound which modulates the said WNK isoform polypeptide protein kinase activity.

The protein kinase activity of the WNK isoform polypeptide that is modulated/assessed in the screening method is phosphorylation of a SPAK or OSR1 polypeptide. Phosphorylation of a SPAK or OSR1 polypeptide may be assessed by measuring activation or phosphorylation of the SPAK or OSR1 polypeptide, as discussed further below and in the Examples. For example, suitable substrates for SPAK or OSR1 polypeptides, which may be used in assessing activation of the SPAK or OSR1 polypeptides, include a fragment of the Na—K—Cl cotransporter (NKCC1) encompassing the N-terminal intracellular domain (residues 1-26), as discussed in the Examples. Methods of measuring phosphorylation of a SPAK or OSR1 polypeptide are also discussed in the Examples. For example, antibodies specific a phosphorylated (or unphosphorylated) phosphorylation site of, for example, SAPK or OSR1, may be used in assessing phosphorylation of that phosphorylation site, as well known to those skilled in the art. Further methods will be apparent to the skilled person on the basis of this teaching and the many known methods of assessing protein phosphorylation.

SPAK or OSR1 activation may also be assessed in a cell using (with suitable controls, as will be apparent to those skilled in the art) a reporter gene whose expression is modulated by SPAK or OSR1, for example a reporter gene that is affected by ion channel activity or cell volume, which are considered to be affected by SPAK or OSR1 activation. SPAK or OSR1 activation may also be assessed in a cell using (with suitable controls, as will be apparent to those skilled in the art) assessments of ion channel activity or cell volume, for example in response to a hyperosmotic shock. Ion channel activity or cell volume may be assessed, as well known to those skilled in the art, by, for example, techniques involving uptake of radioactive ions, or the use of fluorescent dyes.

A second aspect of the invention provides a method for identifying a compound expected to be useful in modulating, for example inhibiting, the activation or phosphorylation of SPAK or OSR1 in a cell, the method comprising the steps of (1) determining whether a test compound modulates, for example inhibits, the protein kinase activity of a WNK isoform polypeptide, and (2) selecting a compound which modulates, for example inhibits, the protein kinase activity of the WNK isoform polypeptide.

The activity of the WNK isoform polypeptide may be measured by measuring the phosphorylation or activation by the WNK isoform polypeptide, in the presence of a suitable phosphate donor, of a SPAK or OSR1 polypeptide, as discussed above. Examples of methods of assessing the phosphorylation or activation of the SPAK or OSR1 polypeptide are indicated above.

The protein kinase activity may be increased or reduced by an alteration in the Vₘₐₓ or the Kₘ (or both) of the WNK isoform polypeptide (or SPAK or OSR1 polypeptide, as appropriate) for a particular substrate. For example, activity may be increased by an increased Vₘₐₓ or decreased Kₘ. It will be appreciated that it may not be necessary to determine the value of either Vₘₐₓ or Kₘ in order to determine whether the WNK isoform polypeptide (or SPAK or OSR1 polypeptide, as appropriate) has been activated or deactivated. It will be appreciated that dephosphorylated (deactivated) SPAK or OSR1 may retain some enzymatic activity (though this is considered to be less than about 1% of the protein kinase activity of the phosphorylated SPAK or OSR1.

Activity may be measured as the amount of a substrate phosphorylated in a given time; a change of activity may therefore be detected as a change in the amount of substrate (for example, at a single concentration) that is phosphorylated in a given time. It is preferred that the activity is increased or decreased, as appropriate, by at least 2, preferably 5, 10, 15, 20, 25, 30 or 50-fold.

It will be appreciated that it may be necessary to determine the effect of the compound on the activity of the substrate (for example SPAK or OSR1), for example by measuring the activity of the substrate when exposed to the compound (1) after exposure of the substrate to the WNK isoform polypeptide, (2) before exposure of the substrate to the WNK isoform polypeptide and/or (3) without exposure to the WNK isoform polypeptide.

By modulation of the protein kinase activity is included inhibition or an increase in the protein kinase activity.

It will be appreciated that in the methods of the invention wherein phosphorylation of a polypeptide may occur that the presence of a suitable phosphate donor may be required, as described for the above aspect of the invention. Suitable phosphate donors will be known to those skilled in the art and include ATP, for example as the magnesium salt (MgATP), as described in the Examples.

The WNK isoform, SPAK and/or OSR1 polypeptides may, for example, be purified from cells in which the WNK isoform, SPAK and/or OSR1 polypeptides are expressed naturally, but it may be more convenient for at least one of the WNK isoform, SPAK and OSR1 polypeptides to be recombinant.

The invention further provides a method of identifying a compound expected to be useful in modulating, for example inhibiting, the activation or phosphorylation of SPAK or OSR1 in a cell, comprising the step of determining the effect of the compound on the ability of a WNK isoform polypeptide to bind to a SPAK polypeptide or OSR1 polypeptide and (2) selecting a compound which modulates, for example inhibits, the ability of said WNK isoform polypeptide to bind to the SPAK or OSR1 polypeptide.

The method may further comprise determining the effect of the compound on the protein kinase activity of the WNK isoform polypeptide or the activation or phosphorylation of SPAK or OSR1, as set out above.

The effect of a compound on ability of a WNK isoform polypeptide to bind to a SPAK polypeptide or OSR1 polypeptide may be measured by any method of detecting/measuring a protein/protein interaction, as discussed further below. Suitable methods include methods such as, for example, yeast two-hybrid interactions, co-purification, ELISA, co-immunoprecipitation and surface plasmon resonance methods. Surface plasmon resonance methods, for example, are well known to those skilled in the art. Techniques are described in, for example, O'Shannessy D J Determination of kinetic rate and equilibrium binding constants for macromolecular interactions: a critique of the surface plasmon resonance literature. Curr Opin Biotechnol. 1994 February; 5(1):65-71; Fivash M, Towler E M, Fisher R J BIAcore for macromolecular interaction. Curr Opin Biotechnol. 1998 February; 9(1):97-101; Malmqvist M BIACORE: an affinity biosensor system for characterization of biomolecular interactions. Biochem Soc Trans. 1999 February; 27(2):335-40.

The term SPAK will be well known to those skilled in the art, as indicated above. The SPAK polypeptide (or other substrate, for example OSR1, NKCC1) used in the assay may be recombinant or non-recombinant. The SPAK may be a bacterially-expressed or mammalian cell-expressed SPAK polypeptide (for example as described in the Examples). The SPAK polypeptide may have the amino acid sequence of a naturally occurring SPAK, or may be or comprise a fusion polypeptide (for example as described in Example 1), or may be a fragment or variant of a naturally occurring SPAK that retains the ability to be phosphorylated or activated by a WNK isoform, for example WNK1 or WNK4, for example as described in Example 1. Thus, it is preferred that the SPAK is an SPAK that retains a threonine (or serine) residue at the position equivalent to Threonine233 of full length wild-type human SPAK. It is preferred that the SPAK is not a mutant in which Thr233 is replaced by a residue other than serine, for example is replaced by alanine. The SPAK may be a SPAK that retains a serine (or threonine) residue at the position equivalent to Serine373 of full length wild-type human SPAK. The SPAK may be not a mutant in which Ser373 is replaced by a residue other than serine, for example is replaced by alanine. A fragment derivable from SPAK which encompasses the Thr233 residue of SPAK and at least part of the T-loop sequence which includes this residue, for example at least the 2, 3, 4, 5, 6 or 7 residues C-terminal and N-terminal of this residue, is a suitable substrate for use in the screening method. A fragment derivable from SPAK which encompasses the Ser373 residue of SPAK and at least part of the sequence which includes this residue, for example at least the 2, 3, 4, 5, 6 or 7 residues C-terminal and/or N-terminal of this residue, is a suitable substrate for use in the screening method.

Examples of Accession numbers for SPAK include NP_{—}037365 (AAC72238, Q9UEW8, AAD01901), NCBI AF099989; IMAGE clone 4825063 and NCBI BG724360.

The term OSR1 will similarly be well known to those skilled in the art, as indicated above. The OSR1 may be a bacterially-expressed or mammalian cell-expressed OSR1 polypeptide (for example as described in the Examples). The OSR1 polypeptide may have the amino acid sequence of a naturally occurring OSR1, or may be or comprise a fusion polypeptide (for example as described in Example 1), or may be a fragment or variant of a naturally occurring OSR1 that retains the ability to be phosphorylated or activated by a WNK isoform, for example WNK1 or WNK4, for example as described in Example 1. Thus, it is preferred that the OSR1 is an OSR1 that retains a threonine (or serine) residue at the position equivalent to Threonine185 of full length wild-type human OSR1. It is preferred that the OSR1 is not a mutant in which Thr185 is replaced by a residue other than serine, for example is replaced by alanine. The OSR1 may be an OSR1 that retains a serine (or threonine) residue at the position equivalent to Serine325 of full length wild-type human OSR1. The OSR1 may be not a mutant in which Ser325 is replaced by a residue other than serine, for example is replaced by alanine. A fragment derivable from OSR1 which encompasses the Thr185 residue of OSR1 and at least part of the T-loop sequence which includes this residue, for example at least the 2, 3, 4, 5, 6 or 7 residues C-terminal and/or N-terminal of this residue, is a suitable substrate for use in the screening method. A fragment derivable from OSR1 which encompasses the Ser325 residue of OSR1 and at least part of the sequence which includes this residue, for example at least the 2, 3, 4, 5, 6 or 7 residues C-terminal and N-terminal of this residue, is a suitable substrate for use in the screening method.

Accession numbers for OSR1 include the following: NCBI_{—}005100 (AAH08726, AAQ02425, BAA75674); IMAGE clone 5271312 (NCBI BI464286). This latter clone has an intronic insertion of 100 nucleotides in the coding region, which can be eliminated by PCR, as discussed in the Examples.

The term WNK isoform will similarly be well known to those skilled in the art. WNK isoforms include WNK1, WNK3 and WNK4, as discussed above. The WNK isoform polypeptide used in the assay may be recombinant or non-recombinant. The WNK isoform polypeptide may be bacterially expressed or expressed in a mammalian system, for example as described in Example 1. It may be appropriate to express the WNK isoform polypeptide alongside the substrate polypeptide, eg a SPAK or OSR1 polypeptide. The WNK isoform polypeptide may have the amino acid sequence of a naturally occurring WNK isoform, or may (for example as described in the Examples) be a fusion polypeptide or may be a fragment or variant of a naturally occurring WNK isoform that retains the ability to phosphorylate or activate SPAK or OSR1, for example on Thr233 or Ser 373 of SPAK, or Thr185 or Ser 325 of OSR1, for example as described in Example 1. Thus, the WNK isoform polypeptide is an WNK isoform polypeptide that retains an active kinase domain. For example, the WNK isoform polypeptide may comprise or consist of residues 1-661 of wild-type human WNK1, or a GST fusion of such a fragment, as described in Example 1. The WNK isoform polypeptide may comprise or consist of residues 1 to 593 of wild-type human WNK4, or a GST fusion of such a fragment, as described in Example 1. A fragment of a WNK isoform which contains the intact kinase domain but not other regions of a WNK isoform may be useful; this region of WNK isoforms is sufficient to retain protein kinase activity. The WNK isoform used in the assay is not a kinase-dead mutant such as is described in the Examples (for example WNK1 in which the residue equivalent to residue D368 of full length human WNK1 is mutated, for example to Alanine; or WNK4 in which the residue equivalent to residue D321 and/or the residue equivalent to K186 of full length human WNK4 is mutated, for example to Alanine). It may be desirable to use as large a fragment of the WNK isoform as possible in order to mimic as closely as possible the behaviour of the naturally occurring WNK isoform.

It is particularly preferred, although not essential, that the WNK isoform polypeptide has at least 30% of the enzyme activity of the full-length human WNK isoform (eg respectively WNK1, WNK2, WNK3 or WNK4) with respect to the phosphorylation of full-length human OSR1 on residue Thr185 or Ser325; or the phosphorylation of full-length human SPAK on residue Thr233 or Ser373; or the phosphorylation of a peptide substrate encompassing such a residue (for example as discussed above). It is more preferred if the WNK isoform polypeptide has at least 50%, preferably at least 70% and more preferably at least 90% of the enzyme activity of the full-length human WNK isoform (eg respectively WNK1, WNK2, WNK3 or WNK4) with respect to the phosphorylation of full-length human OSR1 on residue Thr185 or Ser325; or the phosphorylation of full-length human SPAK on residue Thr233 or Ser373; or the phosphorylation of a peptide substrate encompassing such a residue (for example as discussed above).

Accession numbers for WNK1, WNK2, WNK3 and WNK4 include the following:

| | |
|---|---|
| WNK1: | NP_061852 (CAC15059, Q9H4A3) |
| WNK2: | NM_006648 (NP_006639, BAB21851, CAI12344, CAH73069, |
| | CAI14449, CAB44308, Q9Y3S1, CAH73070 |
| WNK3: | NP_001002838 (Q9BYP7, AAL99253, CAC32455 (and many |
| | others) |
| WNK4: | NP_115763 (AAK91995, Q96J92), CAC48387. |

Similarly, the term NKKC1 will similarly be well known to those skilled in the art. See, for example, NP_{—}001037 (AAP33906, P55011, AAC50561); Liedtke, C. M., Wang, X. and Smallwood, N. D. Role for Protein Phosphatase 2A in the Regulation of Calu-3Epithelial Na⁺—K⁺-2Cl—, Type 1 Co-transport Function J. Biol. Chem. 280 (27), 25491-25498 (2005); Simard, C. F., Daigle, N. D., Bergeron, M. J., Brunet, G. M., Caron, L Noel, M., Montminy, V. and Isenring, P. Characterization of a novel interaction between the secretory Na⁺—K⁺—Cl— cotransporter and the chaperone hsp90 J. Biol. Chem. 279 (46), 48449-48456 (2004). Examples of NKCC1 polypeptides are discussed in Example 1. For example, the NKCC1 polypeptide may be a fragment comprising residues 1-260 of full length NKCC1, or a fusion (for example a GST fusion) of such a fragment. This fragment represents the N-terminal intracellular domain that precedes the first membrane spanning region of NKCC1. The NKCC1 polypeptide may be bacterially expressed, or may be expressed in a mammalian system and/or expressed alongside SPAK or OSR1. The NKCC1 may have the amino acid sequence of a naturally occurring NKCC1, or may be a fusion polypeptide (for example as described in the Examples), or may be a fragment or variant of a naturally occurring NCKK1 that retains the ability to be phosphorylated by SPAK or OSR1. Phosphorylation of NCKK1 by SPAK is discussed in references 28 to 31.

By “variants” of a polypeptide we include insertions, deletions and substitutions, either conservative or non-conservative. In particular we include variants of the polypeptide where such changes do not substantially alter the protein kinase activity or ability to be phosphorylated, as appropriate.

By “conservative substitutions” is intended combinations such as Gly, Ala; Val, Ile, Leu; Asp, Glu; Asn, Gln; Ser, Thr; Lys, Arg; and Phe, Tyr.

The three-letter or one letter amino acid code of the IUPAC-IUB Biochemical Nomenclature Commission is used herein, with the exception of the symbol Zaa, defined above. In particular, Xaa represents any amino acid. It is preferred that at least the amino acids corresponding to the consensus sequences defined herein are L-amino acids.

It is particularly preferred if the polypeptide variant has an amino acid sequence which has at least 65% identity with the amino acid sequence of the relevant human polypeptide, more preferably at least 70%, 71%, 72%, 73% or 74%, still more preferably at least 75%, yet still more preferably at least 80%, in further preference at least 85%, in still further preference at least 90% and most preferably at least 95% or 97% identity with the amino acid sequence of the relevant human polypeptide.

It is still further preferred if a protein kinase variant has an amino acid sequence which has at least 65% identity with the amino acid sequence of the catalytic domain of the human polypeptide, more preferably at least 70%, 71%, 72%, 73% or 74%, still more preferably at least 75%, yet still more preferably at least 80%, in further preference at least 83 or 85%, in still further preference at least 90% and most preferably at least 95% or 97% identity with the relevant human amino acid sequence.

It will be appreciated that the catalytic domain of a protein kinase-related polypeptide may be readily identified by a person skilled in the art, for example using sequence comparisons as described below. Protein kinases show a conserved catalytic core, as reviewed in Johnson et al (1996) Cell, 85, 149-158 and Taylor & Radzio-Andzelm (1994) Structure 2, 345-355. This core folds into a small N-terminal lobe largely comprising anti-parallel β-sheet, and a large C-terminal lobe which is mostly α-helical.

The percent sequence identity between two polypeptides may be determined using suitable computer programs, for example the GAP program of the University of Wisconsin Genetic Computing Group and it will be appreciated that percent identity is calculated in relation to polypeptides whose sequence has been aligned optimally.

The alignment may alternatively be carried out using the Clustal W program (Thompson et al., 1994). The parameters used may be as follows:

Fast pairwise alignment parameters: K-tuple (word) size; 1, window size; 5, gap penalty; 3, number of top diagonals; 5. Scoring method: x percent.

Multiple alignment parameters: gap open penalty; 10, gap extension penalty; 0.05. Scoring matrix: BLOSUM.

The alignment may alternatively be carried out using the program T-Coffee [19], or EMBOSS [20], as discussed in Example 1.

The residue equivalent to, for example, Thr 233 of full-length human SPAK may be identified by alignment of the sequence of the polypeptide with that of full-length human SPAK in such a way as to maximise the match between the sequences. The alignment may be carried out by visual inspection and/or by the use of suitable computer programs, for example the GAP program of the University of Wisconsin Genetic Computing Group, which will also allow the percent identity of the polypeptides to be calculated. The Align program (Pearson (1994) in: Methods in Molecular Biology, Computer Analysis of Sequence Data, Part II (Griffin, A M and Griffin, H G eds) pp 365-389, Humana Press, Clifton). Thus, residues identified in this manner are also “equivalent residues”.

It will be appreciated that in the case of truncated forms of (for example) SPAK or in forms where simple replacements of amino acids have occurred it is facile to identify the “equivalent residue”.

It is preferred that the polypeptides used in the screen are mammalian, preferably human (or a species useful in agriculture or as a domesticated or companion animal, for example dog, cat, horse, cow), including naturally occurring allelic variants (including splice variants). The polypeptides used in the screen may comprise a GST portion or may be biotinylated or otherwise tagged, for example with a 6His, HA, myc or other epitope tag, as known to those skilled in the art, or as described in Examples 1 and 2. This may be useful in purifying and/or detecting the polypeptide(s).

The effect of the compound may be determined by comparing the rate or degree of phosphorylation of the substrate polypeptide by the WNK isoform polypeptide (or the SPAK or OSR1 polypeptide, as appropriate) in the presence of different concentrations of the compound, for example in the absence and in the presence of the compound, for example at a concentration of about 100 μM, 30 μM, 10 μM, 3 μM, 1 μM, 0.1 μM, 0.01 μM and/or 0.001 μM.

A compound identified by a method of the invention may modulate the ability of the WNK isoform polypeptide to phosphorylate different substrates, for example SPAK or OSR1, to different extents. Thus, it is preferred, but not essential, that when screening for a compound for use in modulating SPAK activity that SPAK or a fragment thereof is used as the substrate. Similarly, it is preferred, but not essential, that when screening for a compound for use in modulating the activity of OSR1 that OSR1 or a fragment thereof is used as the substrate.

The method is useful in identifying compounds that, for example, inhibit the activation of SAPK1 and/or OSR1. A compound that inhibits the activation of SAPK1 and/or OSR1 may be useful in the treatment of Gordon's hypertension syndrome or hypertension in general. A compound that promotes the activation of SAPK1 or OSR1 may also be useful, for example in treating low blood pressure, for example following shock.

The compound may be one which binds to or near a region of contact between a WNK isoform and SPAK or OSR1, or may be one which binds to another region and, for example, induces a conformational or allosteric change which stabilises (or destabilises) the complex; or promotes (or inhibits) its formation. The compound may bind to the WNK isoform or to SPAK or OSR1 so as to increase the WNK isoform protein kinase activity by an allosteric effect. This allosteric effect may be an allosteric effect that is involved in the natural regulation of the WNK isoform's activity.

The compounds identified in the methods may themselves be useful as a drug or they may represent lead compounds for the design and synthesis of more efficacious compounds.

The compound may be a drug-like compound or lead compound for the development of a drug-like compound for each of the above methods of identifying a compound. It will be appreciated that the said methods may be useful as screening assays in the development of pharmaceutical compounds or drugs, as well known to those skilled in the art.

The term “drug-like compound” is well known to those skilled in the art, and may include the meaning of a compound that has characteristics that may make it suitable for use in medicine, for example as the active ingredient in a medicament. Thus, for example, a drug-like compound may be a molecule that may be synthesised by the techniques of organic chemistry, less preferably by techniques of molecular biology or biochemistry, and is preferably a small molecule, which may be of less than 5000 daltons. A drug-like compound may additionally exhibit features of selective interaction with a particular protein or proteins and be bioavailable and/or able to penetrate cellular membranes, but it will be appreciated that these features are not essential.

The term “lead compound” is similarly well known to those skilled in the art, and may include the meaning that the compound, whilst not itself suitable for use as a drug (for example because it is only weakly potent against its intended target, non-selective in its action, unstable, difficult to synthesise or has poor bioavailability) may provide a starting-point for the design of other compounds that may have more desirable characteristics.

It will be understood that it will be desirable to identify compounds that may modulate the activity of the protein kinase in vivo. Thus it will be understood that reagents and conditions used in the method may be chosen such that the interactions between, for example, the said WNK isoform polypeptide and, for example, the SPAK or OSR1 polypeptides, are substantially the same as between the human WNK isoform and human SPAK and/or OSR1. It will be appreciated that the compound may bind to the WNK isoform, or may bind to the SPAK or OSR1.

The compounds that are tested in the screening methods of the assay or in other assays in which the ability of a compound to modulate the protein kinase activity of a protein kinase, for example a WNK isoform, may be measured, may be compounds that have been selected and/or designed (including modified) using molecular modelling techniques, for example using computer techniques. The selected or designed compound may be synthesised (if not already synthesised) and tested for its effect on the WNK isoform, for example its effect on the protein kinase activity. The compound may be tested in a screening method of the invention.

It will be appreciated that screening assays which are capable of high throughput operation will be particularly preferred. For example, assays using a substrate peptide based on one of the SPAK or OSR1 phosphorylation sites, for example using an antibody binding to the phosphorylated form of the peptide but not the unphosphorylated for (or vice versa) may be suitable. Examples may include cell based assays and protein-protein binding assays. A further example is an SPA-based (Scintillation Proximity Assay; Amersham International) system as well known to those skilled in the art. For example, beads comprising scintillant and a substrate polypeptide, for example a SPAK or OSR1 peptide substrate as discussed above may be prepared. The beads may be mixed with a sample comprising ³²P or ³³P-γ-labelled ATP, a WNK isoform polypeptide and with the test compound. Conveniently this is done in a 96-well format. The plate is then counted using a suitable scintillation counter, using known parameters for ³²P or ³³P SPA assays. Only p or P that is in proximity to the scintillant, i.e. only that bound to the substrate that is bound to the beads, is detected. Variants of such an assay, for example in which the substrate polypeptide is immobilised on the scintillant beads via binding to an antibody or antibody fragment, may also be used. High throughput protein kinase activity assays are well known to those skilled in the art and can be readily adapted in view of the information provided herein on the phosphorylation of SPAK and OSR1 polypeptides by WNK isoform polypeptides.

The screening method may further comprise the step of assessing whether the compound modulates ion channel, for example NKCC1, activity or cell volume, for example in response to a hyperosmotic stimulus, in a whole cell, tissue or organism; or blood pressure in an organism, and selecting a compound that modulates the activity, cell volume response or blood pressure. The compounds may be tested in whole cells, tissue or organisms that have the WNK1 and/or WNK4 mutations linked to Gordon's syndrome, as discussed above; or that otherwise over-express WNK1, WNK2, WNK3 or WNK4. Ion channel and cell volume response may be measured using techniques involving dyes or radioactive ion takeup, as well known to those skilled in the art.

The screening method may still further comprise the step of assessing whether the compound modulates the activity of a WNK isoform and/or SPAK or OSR1, or the phosphorylation of SPAK, OSR1 and/or NKCC1 in the whole cell, tissue or organism, and selecting a compound that modulates the activity selected.

Compounds may also be subjected to other tests, for example toxicology or metabolism tests, as is well known to those skilled in the art.

The screening method of the invention may comprise the step of synthesising, purifying and/or formulating the selected compound.

The invention also provides a method for preparing a compound which modulates the activity of a WNK isoform or SPAK or OSR1, the method comprising 1) performing a screening method of the invention 2) synthesising, purifying and/or formulating the selected compound.

The compound may be formulated for pharmaceutical use, for example for use in in vivo trials in animals or humans.

A further aspect of the invention is a compound identified or identifiable by a screening method of the invention.

A still further aspect of the invention is a compound of the invention for use in medicine.

The compound may be administered in any suitable way, usually parenterally, for example intravenously, intraperitoneally, subcutaneous or intramuscular or intravesically, in standard sterile, non-pyrogenic formulations of diluents and carriers. The compound may also be administered topically. The compound may also be administered in a localised manner, for example by injection. The treatment may consist of a single dose or a plurality of doses over a period of time. The compound may be useful as an antihypertensive agent or for the treatment of, for example, Gordon's syndrome.

Whilst it is possible for a compound of the invention to be administered alone, it is preferable to present it as a pharmaceutical formulation, together with one or more acceptable carriers. The carrier(s) must be “acceptable” in the sense of being compatible with the compound of the invention and not deleterious to the recipients thereof. Typically, the carriers will be water or saline which will be sterile and pyrogen free.

Thus, the invention also provides pharmaceutical compositions comprising the compound identified or identifiable by the screening methods of the invention and a pharmaceutically acceptable carrier.

The composition may also comprise or be administered with a further compound useful in treating hypertension.

A further aspect of the invention provides a purified preparation comprising a WNK isoform polypeptide and a SPAK and/or OSR1 polypeptide. The preparation may comprise a recombinant WNK isoform polypeptide and/or recombinant SPAK polypeptide and/or OSR1 polypeptide. The preparation may be useful in an assay of the first or second aspect of the invention.

By “purifed” is meant that the preparation has been at least partially separated from other components in the presence of which it has been formed, for example other components of a recombinant cell. Examples of methods of purification that may be used are described in the Examples.

The preparation may be substantially pure. By “substantially pure” we mean that the said polypeptide(s) are substantially free of other proteins. Thus, we include any composition that includes at least 30% of the protein content by weight as the said polypeptides, preferably at least 50%, more preferably at least 70%, still more preferably at least 90% and most preferably at least 95% of the protein content is the said polypeptides.

Thus, the invention also includes compositions comprising the said polypeptides and a contaminant wherein the contaminant comprises less than 70% of the composition by weight, preferably less than 50% of the composition, more preferably less than 30% of the composition, still more preferably less than 10% of the composition and most preferably less than 5% of the composition by weight.

The invention also includes the substantially pure said polypeptides when combined with other components ex vivo, said other components not being all of the components found in the cell in which said polypeptides are found.

A further aspect of the invention provides a recombinant cell capable of expressing a WNK isoform polypeptide and a SPAK polypeptide and/or an OSR1 polypeptide. The cell may comprise a recombinant nucleic acid encoding a WNK isoform polypeptide, and/or a recombinant nucleic acid encoding a SPAK polypeptide, and/or a recombinant nucleic acid encoding an OSR1 polypeptide. The cell may be capable of overexpressing the WNK isoform polypeptide, SPAK polypeptide or OSR1 polypeptide from the endogenous sequence encoding the said polypeptide, for example using techniques of sequence-specific targeting of transcription activators. Thus the cell is modified in a way intended to lead to increased expression of at least one of the WNK isoform, SPAK and OSR1 polypeptide relative to a cell which has not been so modified. The cell may be a prokaryotic or eukaryotic cell. For example it may be a eukaryotic cell, for example an insect, yeast or mammalian cell, for example a human cell. Examples of suitable cells are described, for example, in the Examples.

The recombinant nucleic acid is preferably suitable for expressing the encoded polypeptide. The recombinant nucleic acid may be in the form of an expression vector. Recombinant polynucleotides suitable for expressing a given polypeptide are well known to those skilled in the art, and examples are described in Example 1.

A further aspect of the invention provides a recombinant cell comprising a WNK isoform polypeptide, SPAK polypeptide and/or OSR1 polypeptide. The cell may comprise a recombinant WNK isoform polypeptide and a recombinant SPAK polypeptide or OSR1 polypeptide. The cell may be a cell according to the preceding aspect of the invention. The cell may comprise at least 1.1, 1.2, 1.5, 2, 3, 5, 10 or 20-fold more WNK isoform polypeptide (or SPAK polypeptide, or OSR1 polypeptide, as appropriate) than an equivalent cell which has not been modified in order to overexpress the WNK isoform polypeptide or to express the recombinant WNK isoform polypeptide.

By “suitable for expressing” is mean that the polynucleotide is a polynucleotide that may be translated to form the polypeptide, for example RNA, or that the polynucleotide (which is preferably DNA) encoding the polypeptide of the invention is inserted into an expression vector, such as a plasmid, in proper orientation and correct reading frame for expression. The polynucleotide may be linked to the appropriate transcriptional and translational regulatory control nucleotide sequences recognised by any desired host; such controls may be incorporated in the expression vector.

Characteristics of vectors suitable for replication in mammalian/eukaryotic cells are well known to those skilled in the art, and examples are given below. It will be appreciated that a vector may be suitable for replication in both prokaryotic and eukaryotic cells.

A variety of methods have been developed to operably link polynucleotides, especially DNA, to vectors for example via complementary cohesive termini. Suitable methods are described in Sambrook et al (1989) Molecular Cloning, A Laboratory Manual, Cold Spring Harbor Laboratory, Cold Spring Harbor, N.Y.

A desirable way to modify the DNA encoding a polypeptide of the invention is to use the polymerase chain reaction as disclosed by Saiki et al (1988) Science 239, 487-491. This method may be used for introducing the DNA into a suitable vector, for example by engineering in suitable restriction sites, or it may be used to modify the DNA in other useful ways as is known in the art.

In this method the DNA to be enzymatically amplified is flanked by two specific primers which themselves become incorporated into the amplified DNA. The said specific primers may contain restriction endonuclease recognition sites which can be used for cloning into expression vectors using methods known in the art.

The DNA (or in the case of retroviral vectors, RNA) is then expressed in a suitable host to produce a polypeptide comprising the compound of the invention. Thus, the DNA encoding the polypeptide constituting the compound of the invention may be used in accordance with known techniques, appropriately modified in view of the teachings contained herein, to construct an expression vector, which is then used to transform an appropriate host cell for the expression and production of the polypeptide of the invention. Such techniques include those disclosed in U.S. Pat. Nos. 4,440,859 issued 3 Apr. 1984 to Rutter et al, 4,530,901 issued 23 Jul. 1985 to Weissman, 4,582,800 issued 15 Apr. 1986 to Crowl, 4,677,063 issued 30 Jun. 1987 to Mark et al, 4,678,751 issued 7 Jul. 1987 to Goeddel, 4,704,362 issued 3 Nov. 1987 to Itakura et al, 4,710,463 issued 1 Dec. 1987 to Murray, 4,757,006 issued 12 Jul. 1988 to Toole, Jr. et al, 4,766,075 issued 23 Aug. 1988 to Goeddel et al and 4,810,648 issued 7 Mar. 1989 to Stalker, all of which are incorporated herein by reference.

The DNA (or in the case of retroviral vectors, RNA) encoding the polypeptide may be joined to a wide variety of other DNA sequences for introduction into an appropriate host. The companion DNA will depend upon the nature of the host, the manner of the introduction of the DNA into the host, and whether episomal maintenance or integration is desired.

Generally, the DNA is inserted into an expression vector, such as a plasmid, in proper orientation and correct reading frame for expression. If necessary, the DNA may be linked to the appropriate transcriptional and translational regulatory control nucleotide sequences recognised by the desired host, although such controls are generally available in the expression vector. The vector is then introduced into the host through standard techniques. Generally, not all of the hosts will be transformed by the vector. Therefore, it will be necessary to select for transformed host cells. One selection technique involves incorporating into the expression vector a DNA sequence, with any necessary control elements, that codes for a selectable trait in the transformed cell, such as antibiotic resistance. Alternatively, the gene for such selectable trait can be on another vector, which is used to co-transform the desired host cell.

Host cells that have been transformed by the recombinant DNA of the invention are then cultured for a sufficient time and under appropriate conditions known to those skilled in the art in view of the teachings disclosed herein to permit the expression of the polypeptide, which can then be recovered.

Many expression systems are known, including bacteria (for example E. coli and Bacillus subtilis), yeasts (for example Saccharomyces cerevisiae), filamentous fungi (for example Aspergillus), plant cells, animal cells and insect cells.

The vectors include a prokaryotic replicon, such as the ColE1 ori, for propagation in a prokaryote, even if the vector is to be used for expression in other, non-prokaryotic, cell types. The vectors can also include an appropriate promoter such as a prokaryotic promoter capable of directing the expression (transcription and translation) of the genes in a bacterial host cell, such as E. coli, transformed therewith.

A promoter is an expression control element formed by a DNA sequence that permits binding of RNA polymerase and transcription to occur. Promoter sequences compatible with exemplary bacterial hosts are typically provided in plasmid vectors containing convenient restriction sites for insertion of a DNA segment of the present invention.

Typical prokaryotic vector plasmids are pUC18, pUC19, pBR322 and pBR329 available from Biorad Laboratories, (Richmond, Calif., USA) and pTrc99A and pKK223-3 available from Pharmacia, Piscataway, N.J., USA.

A typical mammalian cell vector plasmid is pSVL available from Pharmacia, Piscataway, N.J., USA. This vector uses the SV40 late promoter to drive expression of cloned genes, the highest level of expression being found in T antigen-producing cells, such as COS-1 cells.

An example of an inducible mammalian expression vector is pMSG, also available from Pharmacia. This vector uses the glucocorticoid-inducible promoter of the mouse mammary tumour virus long terminal repeat to drive expression of the cloned gene.

Useful yeast plasmid vectors are pRS403-406 and pRS413-416 and are generally available from Stratagene Cloning Systems, La Jolla, Calif. 92037, USA. Plasmids pRS403, pRS404, pRS405 and pRS406 are Yeast Integrating plasmids (YIps) and incorporate the yeast selectable markers HIS3, TRP1, LEU2 and URA3. Plasmids pRS413-416 are Yeast Centromere plasmids (YCps).

The host cell can be either prokaryotic or eukaryotic. Bacterial cells are preferred prokaryotic host cells and typically are a strain of E. coli such as, for example, the E. coli strains DH5 available from Bethesda Research Laboratories Inc., Bethesda, Md., USA, and RR1 available from the American Type Culture Collection (ATCC) of Rockville, Md., USA (No ATCC 31343). Preferred eukaryotic host cells include yeast, insect and mammalian cells, preferably vertebrate cells such as those from a mouse, rat, monkey or human fibroblastic cell line. Yeast host cells include YPH499, YPH500 and YPH501 which are generally available from Stratagene Cloning Systems, La Jolla, Calif. 92037, USA. Preferred mammalian host cells include human embryonic kidney 293 cells (see Example 1), Chinese hamster ovary (CHO) cells available from the ATCC as CCL61, NIH Swiss mouse embryo cells NIH/3T3 available from the ATCC as CRL 1658, and monkey kidney-derived COS-1 cells available from the ATCC as CRL 1650. Preferred insect cells are Sf9 cells which can be transfected with baculovirus expression vectors.

Transformation of appropriate cell hosts with a DNA construct is accomplished by well known methods that typically depend on the type of vector used. With regard to transformation of prokaryotic host cells, see, for example, Cohen et al (1972) Proc. Natl. Acad. Sci. USA 69, 2110 and Sambrook et al (1989) Molecular Cloning, A Laboratory Manual, Cold Spring Harbor Laboratory, Cold Spring Harbor, N.Y. Transformation of yeast cells is described in Sherman et al (1986) Methods In Yeast Genetics, A Laboratory Manual, Cold Spring Harbor, N.Y. The method of Beggs (1978) Nature 275, 104-109 is also useful. With regard to vertebrate cells, reagents useful in transfecting such cells, for example calcium phosphate and DEAE-dextran or liposome formulations, are available from Stratagene Cloning Systems, or Life Technologies Inc., Gaithersburg, Md. 20877, USA.

Electroporation is also useful for transforming and/or transfecting cells and is well known in the art for transforming yeast cell, bacterial cells, insect cells and vertebrate cells.

For example, many bacterial species may be transformed by the methods described in Luchansky et al (1988) Mol. Microbiol. 2, 637-646 incorporated herein by reference. The greatest number of transformants is consistently recovered following electroporation of the DNA-cell mixture suspended in 2.5×PEB using 6250V per cm at 25:FD.

Methods for transformation of yeast by electroporation are disclosed in Becker & Guarente (1990) Methods Enzymol. 194, 182.

Successfully transformed cells, ie cells that contain a DNA construct of the present invention, can be identified by well known techniques. For example, cells resulting from the introduction of an expression construct of the present invention can be grown to produce the polypeptide of the invention. Cells can be harvested and lysed and their DNA content examined for the presence of the DNA using a method such as that described by Southern (1975) J. Mol. Biol. 98, 503 or Berent et al (1985) Biotech. 3, 208.

In addition to directly assaying for the presence of recombinant DNA, successful transformation can be confirmed by well known immunological methods when the recombinant DNA is capable of directing the expression of the protein. For example, cells successfully transformed with an expression vector produce proteins displaying appropriate antigenicity. Samples of cells suspected of being transformed are harvested and assayed for the protein using suitable antibodies.

Thus, in addition to the transformed host cells themselves, the present invention also contemplates a culture of those cells, preferably a monoclonal (clonally homogeneous) culture, or a culture derived from a monoclonal culture, in a nutrient medium.

A further aspect of the invention method for making a preparation of the invention, comprising the step of purifying the preparation from a cell according to the invention. Methods of cultivating host cells and isolating recombinant proteins are well known in the art. Examples of suitable purification techniques are described in the Examples. For example, one or more component of the preparation may be tagged so as to aid purification using affinity reagents, as will be well known to those skilled in the art and as described in the Examples. Chromatographic techniques may also be used, for example as described in the Examples.

A further aspect of the invention provides a preparation obtained or obtainable by the method of the preceding aspect of the invention. The preparation may comprise, for example, a tagged WNK isoform polypeptide, SPAK polypeptide or OSR1 polypeptide.

The method of the first or second aspect of the invention may be performed with the WNK isoform polypeptide and SPAK or OSR1 polypeptide in the form of a preparation of the third aspect of the invention; or a preparation or complex obtained or obtainable by the method as indicated above; or in a cell of the invention.

The above polypeptides may be made by methods well known in the art and as described below and in Example 1, for example using molecular biology methods or automated chemical peptide synthesis methods.

It will be appreciated that peptidomimetic compounds may also be useful. Thus, by “polypeptide” or “peptide” we include not only molecules in which amino acid residues are joined by peptide (—CO—NH—) linkages but also molecules in which the peptide bond is reversed. Such retro-inverso peptidomimetics may be made using methods known in the art, for example such as those described in M [symbol] ziΠre et al (1997) J. Immunol. 159, 3230-3237, incorporated herein by reference. This approach involves making pseudopeptides containing changes involving the backbone, and not the orientation of side chains. M [symbol] ziΠre et al (1997) show that, at least for MHC class II and T helper cell responses, these pseudopeptides are useful. Retro-inverse peptides, which contain NH—CO bonds instead of CO—NH peptide bonds, are much more resistant to proteolysis.

Similarly, the peptide bond may be dispensed with altogether provided that an appropriate linker moiety which retains the spacing between the CI atoms of the amino acid residues is used; it is particularly preferred if the linker moiety has substantially the same charge distribution and substantially the same planarity as a peptide bond.

It will be appreciated that the peptide may conveniently be blocked at its N- or C-terminus so as to help reduce susceptibility to exoproteolytic digestion.

Thus, it will be appreciated that the SPAK, OSR1 or NKCC1 polypeptide may be a peptidomimetic compound.

A further aspect of the invention provides a kit of parts comprising a WNK isoform polypeptide or a recombinant polynucleotide encoding a WNK isoform polypeptide, a SPAK polypeptide or a recombinant polynucleotide encoding a SPAK polypeptide, and/or an OSR1 polypeptide or a recombinant polynucleotide encoding an OSR1 polypeptide. Such kits may be useful in forming a preparation or complex which may be useful in, for example a screening method of the first or second aspect of the invention. The recombinant polynucleotide(s) may be in an expression vector (for example as discussed above) or (less desirably) useful for in vitro expression. The SPAK or OSR1 polypeptide may be a peptide encompassing OSR1 residue Thr185 or Ser325; or SPAK residue Thr233 or Ser373.

A further aspect of the invention provides a specific binding partner, typically an antibody, that binds in a phosphorylation state-sensitive manner to an epitope encompassing Thr233 or Ser373 of SPAK (for example human SPAK), or Thr185 or Ser 325 of OSR1 (for example human OSR1). By “binding in a phosphorylation state-sensitive manner” is included the meaning that the specific binding partner is capable of binding to the epitope (or SPAK or OSR1 polypeptide comprising the epitope) when phosphorylated on the phosphorylatable portion, but is not capable of binding to the epitope (or SPAK or OSR1 polypeptide comprising the epitope) when it is not phosphorylated on the phosphorylatable portion of that epitope. Thus, it is preferred that the specific binding partner has at least a 5-fold, preferably 10, 20, 50, 100, 200, 500, 1000, 2000 or 5000-fold difference in affinity for the phosphorylated and non-phosphorylated SPAK or OSR1 substrate polypeptide. In practice, a specific binding partner prepared and purified/selected using methods known in the art (see, for example, WO 03/087400; for example affinity purified using a phosphorylated peptide affinity column and a nonphosphorylated peptide affinity column) is expected to have the required affinity and specificity of binding.

A kit of parts of the invention may further comprise an antibody of the invention.

By the term “antibody” is included synthetic antibodies and fragments and variants (for example as discussed above) of whole antibodies which retain the antigen binding site. The antibody may be a monoclonal antibody, but may also be a polyclonal antibody preparation, a part or parts thereof (for example an Fab fragment or F(ab′)₂) or a synthetic antibody or part thereof. Fab, Fv, ScFv and dAb antibody fragments can all be expressed in and secreted from E. coli, thus allowing the facile production of large amounts of the said fragments. By “ScFv molecules” is meant molecules wherein the V_{H} and V_{L} partner domains are linked via a flexible oligopeptide. IgG class antibodies are preferred.

Suitable monoclonal antibodies to selected antigens may be prepared by known techniques, for example those disclosed in “Monoclonal Antibodies: A manual of techniques”, H. Zola (CRC Press, 1988) and in “Monoclonal Hybridoma Antibodies: techniques and Applications”, JGR Hurrell (CRC Press, 1982), modified as indicated above. Bispecific antibodies may be prepared by cell fusion, by reassociation of monovalent fragments or by chemical cross-linking of whole antibodies. Methods for preparing bispecific antibodies are disclosed in Corvalen et al, (1987) Cancer Immunol. Immunother. 24, 127-132 and 133-137 and 138-143.

A general review of the techniques involved in the synthesis of antibody fragments which retain their specific binding sites is to be found in Winter & Milstein (1991) Nature 349, 293-299.

A further aspect of the invention provides a mutated SPAK polypeptide wherein the T-loop threonine residue corresponding to Thr233 of human SPAK is mutated, for example to an alanine residue or to an aspartate or glutamate residue; and/or wherein the residue corresponding to Ser373 is mutated, for example to an alanine residue or to an aspartate or glutamate residue; and/or wherein the residue corresponding to D212 is mutated, for example to an alanine residue.

A further aspect of the invention provides a mutated OSR1 polypeptide wherein the T-loop threonine residue corresponding to Thr185 of human OSR1 is mutated, for example to an alanine residue or to an aspartate or glutamate residue; and/or wherein the residue corresponding to Ser325 is mutated, for example to an alanine residue or to an aspartate or glutamate residue; and/or wherein the residue corresponding to D164 is mutated, for example to an alanine residue.

A further aspect of the invention provides a peptide comprising a fragment derivable from SPAK which encompasses the Ser373 residue of SPAK or the Thr233 residue of SPAK and at least part of the sequence which includes this residue, for example at least the 2, 3, 4, 5, 6 or 7 residues C-terminal or N-terminal of this residue, wherein the fragment has less than 50, 40, 30, 25, 20, 15 or 10 residues. The peptide may comprise a portion not derivable from SPAK.

A further aspect of the invention provides a peptide comprising a fragment derivable from OSR1 which encompasses the Ser325 residue of OSR1 or the Thr185 residue of OSR1 and at least part of the sequence which includes this residue, for example at least the 2, 3, 4, 5, 6 or 7 residues C-terminal or N-terminal of this residue, wherein the fragment has less than 50, 40, 30, 25, 20, 15 or 10 residues. The peptide may comprise a portion not derivable from OSR1.

A further aspect of the invention provides a polynucleotide encoding a mutated SPAK or OSR1 polypeptide of the invention.

The invention provides a polypeptide comprising the amino acid sequence of human SPAK or OSR1 or a fragment, variant, derivative or fusion thereof wherein the residue equivalent to serine Thr233 (and/or Ser 373 and/or D212) of full-length human SPAK is replaced (for example by an aspartate, glutamate or alanine residue) and/or the residue equivalent to Thr185 (and/or Ser325 and/or D164) of full-length human OSR1 is replaced (for example by aspartate, glutamate or alanine residue). The residue equivalent to Thr233 or Thr185 (or Ser 373 or Ser 325) of full-length human SPAK or OSR1 respectively may be replaced by a residue that is capable of carrying a negative charge (for example an aspartate or glutamate residue), which may mimic the effect of phosphorylation of Thr 233 or Thr 185. Alternatively, these residues may be replaced by a residue, for example alanine, that cannot be phosphorylated and is not capable of carrying a negative charge, and preferably is of a similar bulk to serine or threonine. Replacement of D212A of SPAK or D164 of OSR1 is considered to produce catalytically inactive (kinase dead) polypeptides.

It will be appreciated that phosphorylation of SPAK at Thr233 and/or Ser373 or phosphorylation of OSR1 at Thr185 and/or Ser325 may modulate the activity or, for example, the cellular location of SPAK or OSR1.

It will be appreciated that a polypeptide comprising the amino acid sequence of human SPAK or OSR or a fragment, variant, derivative or fusion thereof wherein the residue equivalent to threonine 233 of full-length human SPAK or threonine 185 of OSR1 is replaced by a residue other than glutamate or aspartate, for example an alanine residue, may not be capable of being activated by phosphorylation, and therefore may not be useful in screening methods of the invention. It will further be appreciated that a SPAK or OSR1 polypeptide wherein the residue equivalent to D212 of full-length human SPAK or D164 of full length human OSR1 is replaced, for example by an alanine residue, is considered not to retain protein kinase activity, and therefore may not be useful in screening methods of the invention in which the effect of compounds on the activation or activity of SPAK or OSR1 is measured. Such SPAK or OSR1 polypeptides may be particularly useful in screening methods of the invention in which phosphorylation of a SPAK or OSR1 polypeptide by a WNK isoform polypeptide is measured, as indicated in the Examples.

A further aspect of the invention is a polynucleotide encoding a polypeptide of the invention. The polynucleotide may be a vector suitable for replication and/or expression of the polypeptide in a mammalian/eukaryotic cell. A still further aspect of the invention is a recombinant polynucleotide suitable for expressing a polypeptide of the invention.

The polynucleotide or recombinant polynucleotide may be DNA or RNA, preferably DNA. The polynucleotide may or may not contain introns in the coding sequence; preferably the polynucleotide is or comprises a cDNA.

A further aspect of the invention provides a method of activating a SPAK polypeptide or OSR1 polypeptide wherein the SPAK polypeptide or OSR1 polypeptide is phosphorylated by a WNK isoform polypeptide, for example a WNK1 or WNK4 polypeptide. The SPAK or OSR1 polypeptide that is activated by the method may be partially or fully deactivated or dephosphorylated SPAK or OSR1 polypeptide.

A further aspect of the invention is a method of reducing the activity of an SPAK or OSR1 polypeptide wherein the SPAK or OSR1 polypeptide is dephosphorylated. The SPAK or OSR1 polypeptide the activity of which is reduced may be a partially or fully activated/phosphorylated SPAK or OSR1 polypeptide. SPAK or OSR1 may be dephosphorylated by a serine/threonine protein phosphatase, for example PP1 (Berndt et al (1987) FEBS Lett 223, 340-346), PP2C (Mann et al (1992) Biochem et Biophys Acta 1130, 100-104 or protein phosphorylase 2A (PP2A; da Cruz E Silva (1987) FEBS Lett 221, 415-422) or a variant, fragment, fusion or derivative any thereof, or a fusion of a said variant, fragment or derivative. PP1, PP2C or PP2A or a variant, fragment, fusion or derivative any thereof, or a fusion of a said variant, fragment or derivative is capable of dephosphorylating phosphorylase α. The terms PP1, PP2C, PP2A and phosphorylase α are well known to those skilled in the art. PP2A may be inactivated by microcystin-LR at 1 μM. SPAK or OSR1 synthesised in bacterial cells is considered to be unphosphorylated. Phosphorylation state may be judged using mass spectrometry.

As for preceding aspects of the invention, by “activating” is meant that the enzymatic activity of, for example, the SPAK or OSR1 polypeptide is increased. By “reducing the activity” is meant that the enzymatic activity of the SPAK or OSR1 polypeptide is reduced. The enzymatic activity that may be increased or reduced is protein kinase activity, preferably Serine/Threonine protein kinase activity ie the phosphorylation of a protein/polypeptide on one or more serine or threonine residues. The enzymatic activity may be increased or reduced by an alteration in the Vₘₐₓ or the Kₘ (or both) of the SPAK or OSR1 polypeptide for a particular substrate, for example NKCC1 or a fragment thereof, for example as discussed above or indicated in the Examples. For example, activity may be increased by an increased Vₘₐₓ or decreased Kₘ. It will be appreciated that it may not be necessary to determine the value of either Vₘₐₓ or Kₘ in order to determine whether the SPAK or OSR1 polypeptide has been activated or deactivated. It will be appreciated that dephosphorylated (deactivated) SPAK or OSR1 polypeptide may retain some enzymatic activity.

Activity may be measured as the amount of a substrate phosphorylated in a given time; a change of activity may therefore be detected as a change in the amount of substrate (for example, at a single concentration) that is phosphorylated in a given time, as described in Example 1.

It will be appreciated that if the SPAK or OSR1 polypeptide is already phosphorylated, further phosphorylation may not be possible and/or may not lead to further activation. Further, if the SPAK or OSR1 polypeptide is already partially dephosphorylated, then further dephosphorylation may not be possible and/or may not lead to further deactivation.

It will further be appreciated that SPAK or OSR1 polypeptide isolated from cells (either as an endogenous or recombinant polypeptide) may be heterogeneous with regard to its phosphorylation/activation state. For example, fully activated/phosphorylated, fully deactivated/dephosphorylated and/or partially activated/phosphorylated molecules of SPAK or OSR1 polypeptides may be present in a single cell or group/culture of cells.

It is preferred that the activity is increased or decreased, as appropriate, by at least 2, preferably 5, 10, 15, 20, 25, 30 or 50-fold.

A further aspect of the invention provides the use of a WNK isoform polypeptide in a method of activating and/or phosphorylating a SPAK polypeptide or OSR1 polypeptide. The SPAK or OSR1 polypeptide may be a dephosphorylated SPAK or OSR1 polypeptide, as discussed above.

The OSR1 polypeptide may be phosphorylated on the T-loop residue corresponding to Thr185 of full length human OSR1. The SPAK polypeptide may be phosphorylated on the T-loop residue corresponding to Thr233 of full length human SPAK.

The OSR1 polypeptide may further or alternatively be phosphorylated on the residue equivalent to Ser325 of full-length human OSR1. The SPAK polypeptide may be further phosphorylated on the residue equivalent to Ser373 of full length human SPAK.

The invention will now be described in more detail by reference to the following, non-limiting, Figures and Examples.

Any documents referred to herein are hereby incorporated by reference.

### FIGURE LEGENDS

FIG. 1. Analysis of WNK1 binding proteins. A. Rat tissue extracts (40 μg protein) were immunoblotted with the indicated antibodies. Similar findings were obtained in at least 2 separate experiments. B. Rat testis extracts were subjected to immunoprecipitation with WNK1 or pre-immune antibody. The immunoprecipitates were electrophoresed on a polyacrylamide gel and the protein bands visualised following colloidal Coomassie blue staining. The major bands observed in the WNK1 but not the pre-immune purification are indicated. C These bands as well as the corresponding region in the pre-immune sample were excised from the gel, digested in gel with trypsin, and identities of the proteins determined by tryptic peptide mass-spectral fingerprint as described in the experimental procedures. Mascot protein score where a value of >63 is considered significant (p<0.05), number (No.) of peptides sequenced by MALDI-TOF/TOF MS/MS (SPAK and SDB84) or LC MS/MS (WNK1) and accession numbers for each protein identified are indicated. Tryptic peptides derived from WNK1, WNK3, SPAK and SDB84, were present in the WNK1 but not pre-immune immunoprecipitate.

FIG. 2. Interaction of WNK1/WNK4 with SPAK and OSR1. A. Multiple sequence alignment of the indicated STE20 human kinases. Identical residues are highlighted in black and similar residues in grey. The kinase domain is marked with a solid line, regions of non-similarity between SPAK and OSR1 are marked with a dotted line, the magnesium binding Asp residue mutated to inactivate SPAK and OSR1 is marked with a triangle, the T-loop Thr phosphorylated by WNK1/WNK4 is marked with an asterisk, and the non-catalytic Ser phosphorylated by WNK1/WNK4 with a square. B. HEK 293 cells were transfected with constructs encoding the indicated GST-fusion proteins. 36 h post transfection, the GST-fusion proteins were affinity purified and immunoblotted with GST antibodies or WNK1 antibody to detect endogenously associated WNK1. C HEK 293, these were co-transfected with the indicated combinations of FLAG tagged WNK1/WNK4, GST-SPAK, GST-OSR1 or empty pEBG2T vector. GST-fusion proteins were affinity purified and immunoblotted with FLAG antibody to detect WNK1 or WNK4 expression or GST antibody to detect SPAK, OSR1 or GST expression.

FIG. 3. Phosphorylation of SPAK/OSR1 by WNK1 and WNK4. A. Kinase active wild type (wt) WNK1[1-661] or kinase-inactive (ki) WNK1[1-661/D368A] was incubated with the indicated proteins (ki-SPAK[D212A], ki-OSR1[D164A], MBP, claudin-4 and H2A) in the presence of Mg²⁺ and [γ³²P]ATP. Phosphorylation of protein substrates was determined following electrophoresis on polyacrylamide gel and autoradiography (upper panel) of the Coomassie blue stained bands (lower panel) corresponding to each substrate. B. As above expect that kinase active wild type (wt) WNK4[1-593] or ki-WNK4[1-593/K186A/D321A] were employed. A double mutant of WNK4 was generated in order to ensure complete catalytic inactivation of the kinase. For both A & B each experimental condition was assayed in duplicate and similar results obtained in at least 2 different experiments.

FIG. 4. Activation of SPAK/OSR1 by WNK1 and WNK4. A. The indicated combinations of wild type (wt) or kinase inactive (ki) WNK1[1-661] were tested for their ability to activate wt-SPAK or ki-SPAK[D212A]. Activity of SPAK was assayed employing NKCC1[1-260] as a substrate. Phosphorylation of NKCC1 was quantified following electrophoresis on polyacrylamide gel and autoradiography (middle panel) of the Coomassie blue stained band of NKCC1[1-260] (lower panel). The phosphorylation of NKCC1 was also measured as ³²P-radioactivity by Cerenkov counting (upper panel). The results are plotted as average ±SD of a duplicate experiment, relative to the phosphorylation obtained with wt-SPAK alone. B As above except that wt-OSR1 or ki-OSR1[D164A] were employed. C and D as above except that wt-WNK4[1-593] or ki-WNK4[1-593/K186A/D321A] were employed.

FIG. 5. Analysis of phosphorylation of SPAK and OSR1. A. kinase-inactive SPAK[D212A] was phosphorylated by wild type WNK1[1-661] for 40 min under conditions in which phosphorylation was maximal (data not shown). The ³²P-labelled SPAK was isolated by electrophoresis on a polyacrylamide gel, digested with trypsin and the resulting peptides were chromatographed on a C₁₈ column. Fractions containing the major ³²P-labelled peptides are marked. B. The indicated peptides were analysed by MALDI TOF-TOF mass spectrometry as described in the Materials and Methods. The site of phosphorylation within each peptide was determined by solid phase Edman sequencing in which ³²P-radioactivity was measured after each cycle of Edman degradation. The cycle number in which ³²P-radioactivity was released is indicated. The deduced amino acid sequences of P₁, P₂, P₃, and P₄ are indicated in which the phosphorylated residues are underlined. C and D as above except that kinase-inactive OSR1[D164A] was employed.

FIG. 6. Analysis of phosphorylation of OSR1 by WNK1 and WNK4. A. The indicated mutants of the kinase-inactive (ki) OSR1[D164A] were phosphorylated by wild type WNK1[1-661] in the presence of Mg²⁺ and [γ³²P]ATP. Phosphorylation of OSR1 was determined following electrophoresis on polyacrylamide gel and autoradiography (upper panel) of the Coomassie blue stained band (lower panel) corresponding to OSR1. B. As above except that phosphorylation reactions were terminated at the indicated time points and the stoichiometry of ³²P-phosphorylation of the indicated mutants of ki-OSR1 was determined by Cerenkov counting. The results are plotted as average ±SD of a duplicate experiment. Abbreviations: ki, kinase-inactive OSR1 [D164A]; TA, OSR1[D164A/T185A]; SA, OSR1 [D164A/S325A], TASA, OSR1[D164A/T185A/S325A]. C. As in A. except WNK4[1-593] was employed.

FIG. 7. Analysis of activation of OSR1 by WNK1. The indicated mutants of OSR1 were incubated in the absence (−) or presence (+) of WNK1[1-661] in the presence of Mg²⁺ and [γ³²P]ATP. Following this incubation, the activity of OSR1 was assayed employing NKCC1[1-260] as a substrate. Phosphorylation of NKCC1 was quantified following electrophoresis on polyacrylamide gel and autoradiography (middle panel) of the Coomassie blue stained band of NKCC1[1-260] (lower panel). The phosphorylation of NKCC1 was also measured as ³²P-radioactivity by Cerenkov counting (upper panel). The results are plotted as average ±SD of a duplicate experiment, relative to the phosphorylation obtained with wt-OSR1 alone. Abbreviations: wt, wild type; ki, kinase-inactive, OSR1[D164A]; TA, OSR1[T185A]; TE, OSR1[T185E]; SA, OSR1[S325A]; SE, OSR1[S325E]; TASA, OSR1[T185A/S325A]; TESE, OSR1[T185E/S325E].

FIG. 8. Summary of the mechanism by which hyperosmotic stresses may stimulate NKCC1 cotransporter activity.

FIG. 9. Measurement of WNK1 kinase activity using SPAK peptides as the substrates. Timecourses of incorporation in the presence of GST-WNK1 (61-667) of ³²P-phosphate into substrates OSR1 [D164A], T233 SPAK peptide substrate and S373 PSAK peptide substrate are shown. Details are given in Example 3.

FIG. 10. WNK dependent labelling of OSR1(D164A) 100 μM ATP, 1 h incubation. Details are given in Example 4.

FIG. 11. ATP dose response 75 ng WNK1, 20 min incubation. Details are given in Example 4.

FIG. 12. Hypericin Inhibition of WNK1. Details are given in Example 4.

### EXAMPLE 1

### Phosphorylation and Activation of the STE20 Family Kinases SPAK and OSR1 by the WNK1 and WNK4 Protein Kinases

Abstract. Mutations in the human genes encoding With No lysine Kinase-1 (WNK1) and the related protein kinase WNK4, are the cause of Gordon's hypertension syndrome. Little is known about the molecular mechanism by which WNK isoforms regulate cellular processes. We immunoprecipitated WNK1 from extracts of rat testis and found that it was specifically associated with a protein kinase of the STE20 family, termed STE20/SPS1-related Proline-Alanine-rich Kinase (SPAK). We demonstrated that WNK1 and WNK4 both interacted with SPAK as well as a closely related kinase, termed Oxidative Stress Response kinase-1 (OSR1). Wild type, but not catalytically inactive WNK1 and WNK4, phosphorylated SPAK and OSR1, to a greater extent than other substrates utilised previously, such as myelin basic protein and claudin-4. Phosphorylation by WNK1 or WNK4 markedly increased SPAK and OSR1 activity. Phosphopeptide mapping studies demonstrated that WNK1 phosphorylated kinase-inactive SPAK and OSR1 at an equivalent residue located within the T-loop of the catalytic domain (Thr233-SPAK, Thr185-OSR1) and a Ser residue located within a C-terminal non-catalytic region (Ser373-SPAK, Ser325-OSR1). Mutation of Thr185 to Ala, prevented the activation of OSR1 by WNK1, while mutation of Thr185 to Glu to mimic phosphorylation, increased the basal activity of OSR1 over 20-fold, and prevented further activation by WNK1. Mutation of Ser325 in OSR1 to Ala or Glu did not affect the basal activity of OSR1 or its ability to be activated by WNK1. These findings suggest that WNK isoforms operate as protein kinases that activate SPAK and OSR1 by phosphorylating the T-loops of these enzymes resulting in their activation. Our analysis also describes the first facile assay that can be employed to quantitatively assess WNK1 and WNK4 activity.

Abbreviations: GST, glutathione-S-transferase; H2A, Histone 2A; Id, kinase inactive; MBP, myelin basic protein; NKCC1, Na—K—Cl-Cotransporter-1; OSR1, Oxidative Stress Response kinase-1; PHAII, pseudohypoaldosteronism type II; SPAK, STE20/SPS1-related Proline-Alanine-rich Kinase; WNK, With No K(lysine) protein kinase.

### Materials and Methods.

Materials. Sequencing grade trypsin, DNAase1 and protease-inhibitor cocktail tablets Complete were from Roche; dialysed fetal bovine serum and other tissue culture reagents were from Life Technologies; [γ³²P]ATP, Protein G Sepharose, Glutathione-Sepharose 4B and enhanced chemiluminescence were from Amersham Biosciences; Myelin basic Protein (MBP), the precast 10% and 4-12% Bis-Tris SDS-polyacrylamide gels and 3-8% Tris-Acetate were from Invitrogen. Tween-20, 4-vynilpyridine, rabbit IgG-Agarose and dimethyl pimelimidate were from Sigma Lysozyme from hen egg and NP-40 were from Fluka; GST-PreScission Protease was expressed and purified from E. coli using plasmids kindly provided by John Heath (Birmingham) and David Barford (London). Human H2A expressed and purified from E. coli was kindly provided by Nicola Wiechens and Tom Owen-Hughes (School of Life Science, Dundee, Scotland). All peptides were synthesised by Dr. Graham Bloomberg at the University of Bristol.

Antibodies. The WNK1-(Total) antibody was raised in sheep against WNK1 protein encompassing residues 61-661 expressed in E. coli, the WNK1-(CT) was raised in sheep against residues 2360-2382 of human WNK1, QNFNISNLQKSISNPPGSNLRTT and affinity purified on the peptide antigen [13]. Total ERK1/2 (#9102) antibody was purchased from Cell Signalling Technology. Mouse monoclonal antibodies anti-FLAG M2 (#F3165) and mouse monoclonal antibodies recognising GST were purchased from Sigma (#G1160). Secondary antibodies coupled to horseradish peroxidase were from Pierce.

General methods and buffers. Tissue culture, transfection, immunoblotting, restriction enzyme digests, DNA ligations, and other recombinant DNA procedures were performed using standard protocols. All mutagenesis were carried out using the Quick-Change site-directed mutagenesis method (Stratagene). DNA constructs used for transfection were purified from E. coli DH5α using Qiagen plasmid Mega or Maxi kit according to the manufacturer's protocol. All DNA constructs were verified by DNA sequencing, which was performed by The Sequencing Service, School of Life Sciences, University of Dundee, Scotland, UK, using DYEnamic ET terminator chemistry (Amersham Biosciences) on Applied Biosystems automated DNA sequencers. Rat tissues were rapidly excised and frozen in liquid nitrogen, lysed and stored at −80° C., as described previously [14]. Lysis Buffer: 50 mM Tris-HCl pH 7.5, 1 mM EDTA, 1 mM EGTA, 1% (by mass) NP-40, 1 mM sodium orthovanadate, 50 mM sodium fluoride, 5 mM sodium pyrophosphate, 0.27 M sucrose, 1 mM DTT and ‘Complete’ protease inhibitor cocktail (one tablet per 50 ml). Buffer A was 50 mM Tris-HCl pH 7.5, 0.1 mM EGTA, 1 mM DTT. Sample Buffer 2% (by mass) LDS, 10% (by vol) glycerol, 0.2 M Tris pH 8.5, 25 mM DTT and 0.5 mM EDTA. DNA constructs. The cloning of human WNK1 [13], STRADα and STRADβ[15], were described previously. A full-length human WNK4 (NCBI AAK91995) cDNA was obtained by combing EST NCBI CA388932 (EST1, kindly provided by G. Wistow, National Eye Institute, NIH, Bethesda) and IMAGE clone 2583218 (EST2, NCBI AW082836, ordered from the IMAGE Consortium). Residues 1-680 with an N-terminal BamnH1-site and FLAG-tag were amplified by PCR from EST1, while residues 681-1243 was amplified from EST2. The full-length cDNA clone was assembled in the pEBG2T vector, using BamH1-Spe1-Spe1-Not1 3-way ligation. Full length WNK4 was subcloned into the pCMV5 vector as a BamH1H-Not1 and residues encompassing 1-593 were amplified by PCR and subcloned into pEBG2T in order to express an active fragment of WNK4 in mammalian cells. The coding region of human SPAK (NCBI AF099989) was amplified from IMAGE clone 4825063 (NCBI BG724360) with an N-terminal HA tag and subcloned into pEBG6P and pGEX-6P-1 (Amersham) expression vectors as a BamH1-BamH1 fragment. Human OSR1 (NCBI NP_{—}005100) was cloned from the IMAGE clone 5271312 (NCBI BI464286). This EST possessed an intronic insertion of 100 nucleotides in the coding region, which was eliminated by PCR. Full length OSR1 with an N-terminal HA-tag was amplified and subcloned as a BamH1-Not1 fragment into pEBG6P and pGEX-6P-1 expression vectors. Human claudin-4 (NCBI NP_{—}001296) with an N-terminal HA tag was amplified from EST IMAGE clone 3349211 (NCBI: AAS07556) and after sequencing it was subcloned as a BamH1-Not1 fragment into pEBG2T and pGEX-6P-1 expression vectors. A cDNA coding for the shark NKCC1 cotransporter (NCBI P55013) was kindly provided by Florian Lang (University of Tübingen). Residues 1-260 of NKCC1 were amplified by PCR and subcloned as a BamH1-Not1 fragment into pGEX-6P-1 expression vector.

Immunoblotting. Samples were heated in SDS sample buffer, subjected to SDS-polyacrylamide gel electrophoresis and transferred to nitrocellulose. Membranes were blocked for 5 min in 50 mM Tris/HCl pH 7.5, 0.15 M NaCl, 0.2 (by vol) % Tween-20 (TBST), containing 10% (by mass) skimmed milk. The membranes were then incubated for 16 h at 4° C. with 1 μg/ml of the sheep antibodies or 1000-fold dilution for commercial antibodies in TBST containing 10% (by mass) skimmed milk for the sheep, mouse antibodies and 5% (by mass) bovine serum albumin for commercial anti ERK1/2 antibody. Detection of proteins was performed using horse radish peroxidase conjugated secondary antibodies and the enhanced chemiluminescence reagent.

Immunoprecipitation of endogenous WNK1. The WNK1-(CT) and pre-immune IgG antibodies were covalently coupled to protein G-Sepharose in a ratio of 1 mg of antibody to 1 ml of resin using a dimethyl pimelimidate cross-linking procedure [16]. As a pre-clearing step, 50 mg of rat testis lysate was incubated at 4° C. for 20 min on a rolling shaker with 0.5 ml of protein G-Sepharose. The supernatant was then incubated at 4° C. for 1 h on a rolling shaker with 50 μl of WNK1-(CT) or IgG-protein G-Sepharose conjugated antibodies. The immunoprecipitates were washed four times with 10 ml of Lysis Buffer containing 0.15 M NaCl and lacking DTT and twice with 10 ml of 10 mM Tris/HCl pH 8, 0.1 mM EGTA. The resin was resuspended in total volume of 0.1 ml and 30 μl of NUPAGE-LDS Sample Buffer (purchased from Invitrogen containing 0.14 M Tris 2% (w/v) LDS (lithium dodecyl sulphate), 10% (v/v) glycerol final pH 8.5) in the absence of DTT. The samples were filtered through a 0.44 μm Spin-X filter tube (Coring), DTT added to a final concentration of 10 mM, samples were heated for 5 min at 70° C. and concentrated by speed-vacuum centrifugation to 30 μl. Samples were alkylated for 30 min at room temperature using 50 mM 4-vynilpyridine in 10 mM NH₄HCO₃ and then subjected to electrophoresis on a 4-12% gel using MOPS as a running buffer. The gel was stained with Colloidal Blue Coomassie (Invitrogen). The bands were excised, washed and digested with trypsin as described previously [17]. Peptides were analysed by combined MALDI-TOF, MALDITOF/TOF MS (matrix-assisted laser-desorption ionization-time-of-flight/time of flight mass spectrometry) analysis on an Applied Biosystems 4700 ToF/ToF Proteomics Analyser using 5 mg/ml alpha cyano-cinnamic acid in 10 mM ammonium phosphate as the matrix or by LC-MS on an Applied Biosystems 4000 Q-TRAP. The Celera Discovery System (Applied Biosystems) human database was searched using the Mascot search algorithm (http:www.matrixscience.com [18]).

Multiple sequence alignment. The protein Sequence alignments of human SPAK, OSR1, STRADα and STRADβ were performed using the program T-Coffee [19]. The alignment was graphically represented with BOXSHADE version 3.21 at http://www.ch.embnet.org/software/BOX_form.html using standard parameters. The pairwise alignments and the calculation of the percentage of sequence identity were performed with EMBOSS (parameters: Matrix Blosum50; open gap penalty 10; gap extension penalty 0.5) [20]

Expression and purification GST fusion proteins in human embryonic kidney 293 cells. 10 cm diameter dishes of HEK293 cells were transfected with 5-10 μg of the pEBG-6P or pEBG-2T constructs alone or together with the indicated pCMV5 constructs using the polyethylenimine method [21]. 36 hours post-transfection, the cells were lysed 0.5 ml of ice-cold Lysis Buffer and the clarified lysates were incubated for 1 hour on a rotating platform with glutathione-Sepharose 4B (10 μl/dish of lysate) previously equilibrated in Lysis Buffer. The beads were washed four times with Lysis Buffer containing 0.15 M NaCl and two times with Buffer A containing 0.27 M sucrose. The resin was incubated in a 1:1 slurry of Buffer A containing 0.27 M sucrose and 20 mM glutathione to elute the GST-fusion proteins. The beads were then removed by filtration through a 0.44 μm Spin-X filter tube (Corning) and the eluate divided into aliquots, snap frozen in liquid nitrogen and stored at −80° C.

Expression of WNK1[1-661], SPAK, OSR1, NKCC1[1-260] and claudin-4 in E. coli.

All pGEX-6P-1 constructs were transformed into BL21 E. coli cells and a 0.5 L culture was grown at 37° C. in Luria Broth containing 100 μg/ml ampicillin and 30 μg/ml chloramphenicol until the absorbance at 600 nm was 0.8 and 30 μM isopropyl-β-D-thiogalactopyranoside was added. The cells were cultured for a further 8 to 16 hours at 26° C., resuspended in 12.5 ml of ice-cold Lysis Buffer and frozen in liquid nitrogen. After thawing, 12.5 ml of Lysis Buffer containing 1 mg/ml chicken egg lysozyme and 10 U/ml of DNAase1 were added. Samples were gently agitated at 4° C. for 20 minutes and then sonicated briefly. Lysates were centrifuged at 4° C. for 15 min at 26,000×g and incubated with 0.5 ml of glutathione-Sepharose for 1 hour. The resin was washed in Lysis Buffer containing 0.5 M NaCl followed by Buffer A containing 0.27 M sucrose. Proteins were either eluted by addition of 20 mM glutathione or by incubating the resin overnight with GST-PreScission Protease (30 μg/ml of slurry). Eluted protein was stored in aliquots in −80° C.

In vitro WNK1 and WNK4 phosphorylation reactions. Assays were set up in a total vol of 25 μl of buffer A containing: 0.1 μM of the kinase (GST-WNK1[1-661] or GST-WNK1[1-661/D368A] or GST-WNK4[1-593] or GST-WNK4[1-593/K186A/D321A]), 5 μM of substrate (SPAK[D212A] or OSR1 [D164A] or MBP or Claudin-4 or H2A), 10 mM MgCl₂, 0.1 mM [γ³²P]ATP (˜300 cpm/pmole). After incubation for 30 min at 30° C., incorporation of phosphate was determined following electrophoresis of samples on a NuPAGE Bis-Tris 10% gels and autoradiography of the dried Coomassie-stained gels. The proteins band corresponding to SPAK, OSR1, MBP, Claudin-4 and H2A were excised and phosphate incorporation was quantified on a Wallac Scintillation Counter.

Activation of SPAK/OSR1 by WNK1/WNK4. The activation assays mix were set up in a total vol of 25 μl of Buffer A containing: 0.25 μM of the kinase (GST-WNK1[1-661] or GST-WNK1[1-661/D368A] or GST-WNK4[1-593] or GST-WNK4[1-593/K186A/D321A]) 5 μM of substrate (GST-SPAK or GST-SPAK[D212A] or GST-OSR1 or GST-OSR1 [D164A]), 10 mM MgCl₂, 0.1 mM non-radioactive ATP. After incubation for 40 min at 30° C., 5 μl of the activation assay mix was transferred to a 201 solution containing 6.25 μM NKCC1[1-260], 10 mM MgCl₂, 0.1 mM [γ³²P]ATP (˜300 cpm/pmole). After incubation for 20 min at 30° C. incorporation of phosphate was determined following electrophoresis of samples on a NuPAGE Bis-Tris 10% gels and autoradiography of the dried Coomassie-stained gels. The protein bands corresponding to NKCC1 were excised and phosphate incorporation was quantified on a Wallac Scintillation Counter. It should be noted that NKCC1[1-260] (in which the GST tag has been removed) migrates with an apparent molecular mass of 40 kDa on our gel system. Electrospray mass spectrometry confirmed that the molecular mass of the NKCC1[1-260] fragment corresponded to the theoretical mass (data not shown).

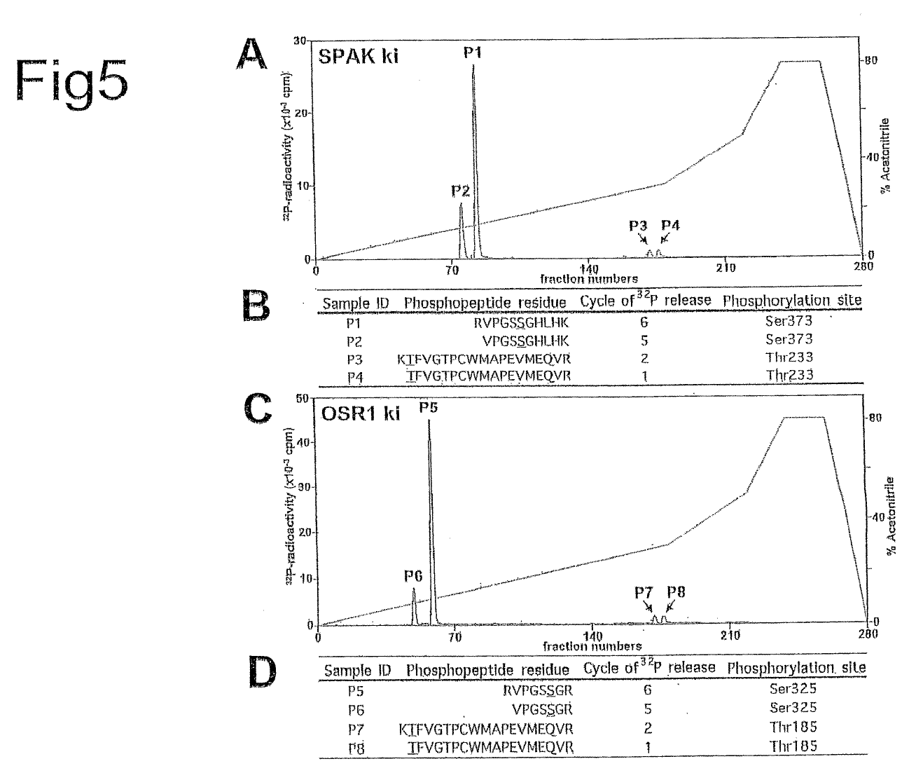

Mapping phosphorylation sites on SPAK and OSR1. The phosphorylation assays were set up in a vol of 25 μl of Buffer A containing: 2 μM GST-WNK1[1-661], 10 μM SPAK[D212A] or OSR1[D164A], 10 mM MgCl₂, 0.1 □M [γ³²P]ATP (˜2000 cpm/pmole). After incubation for 40 min at 30° C., incorporation of phosphate was determined following electrophoresis of samples on a NuPAGE Bis-Tris 10% gels and autoradiography of Coomassie-stained gels. The proteins band corresponding to SPAK and OSR1 were excised and phosphate incorporation was quantified on a Wallac Scintillation Counter. Following tryptic digestion more than >86% of the ³²P-radioactivity incorporated in the gel band was recovered and the samples were chromatographed on a reverse phase HPLC Vydac 218TP5215 C₁₈ column as described in the legend of FIG. 5. Peptides analysis of the fractions corresponding to the major ³²P-containing peaks were analysed using an Applied Biosystems 4700 Proteomics Analyser (MALDI-TOF-TOF) and solid-phase Edman degradation on an Applied Biosystems 494C sequenator of the peptide coupled to Sequelon-AA membrane (Milligen) as described previously [22].

### Results.

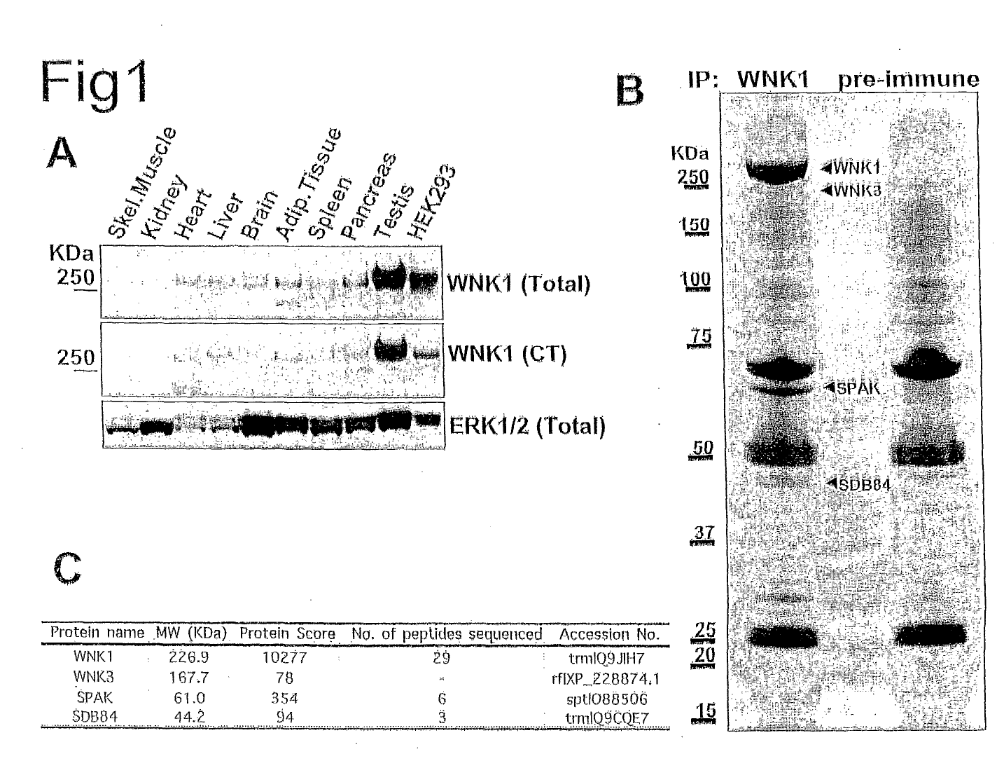

Analysis of WNK1 interacting proteins. In order to identify a tissue in which WNK1 was highly expressed, we immunoblotted adult rat tissue extracts, with two anti-WNK1 antibodies (FIG. 1A). We found that WNK1 was expressed in several tissues, but was most abundant in testis. We were unable to detect significant expression of WNK1 in skeletal muscle or kidney extracts (FIG. 1A). WNK1 was also expressed at a high level in human embryonic kidney 293 epithelial cells, albeit at a lower level than in testis. In order to identify WNK1-binding proteins, we immunoprecipitated endogenous WNK1 from 50 mg of testis extract and, in parallel experiments, performed a control immunoprecipitation using a pre-immune antibody. The purified preparations were subjected to electrophoresis on a polyacrylamide gel, which was then stained with colloidal Coomassie blue (FIG. 1B). The identities of the major bands in the WNK1 and control immunoprecipitation were established by tryptic peptide mass-spectral fingerprinting procedures (FIG. 1C & data not shown). In the WNK1 immunoprecipitation, the major 250 kDa band that was not present in the control immunoprecipitates, was identified as WNK1. In addition to WNK1, one major colloidal Coomassie blue-stained band at 65 kDa was also only observed in the WNK1, but not in the control immunoprecipitation, was identified as SPAK, a STE20-like kinase [23]. Two other more minor bands that appeared specifically associated with WNK1 immunoprecipitate were identified as WNK3 (˜220 kDa) [2] and SDB84 antigen (˜45 kDa) [24]. WNK3 is unlikely to be directly immunoprecipitated with the WNK1 antibody raised against the C-terminal peptide of WNK1, as the sequence to which the antibody was raised is not conserved in WNK3. However, as WNK1 and WNK4 have been reported to oligomerise [25], WNK3 might also bind WNK1. WNK3 could also be bound to the SPAK polypeptide. We cloned SDB84, but were unable to demonstrate that it interacted with WNK1 when overexpressed in HEK293 cells, suggesting that it may not interact with WNK1 directly. We were also unable to demonstrate that WNK1 phosphorylated recombinant SDB84 in a standard in vitro kinase assay (data not shown).

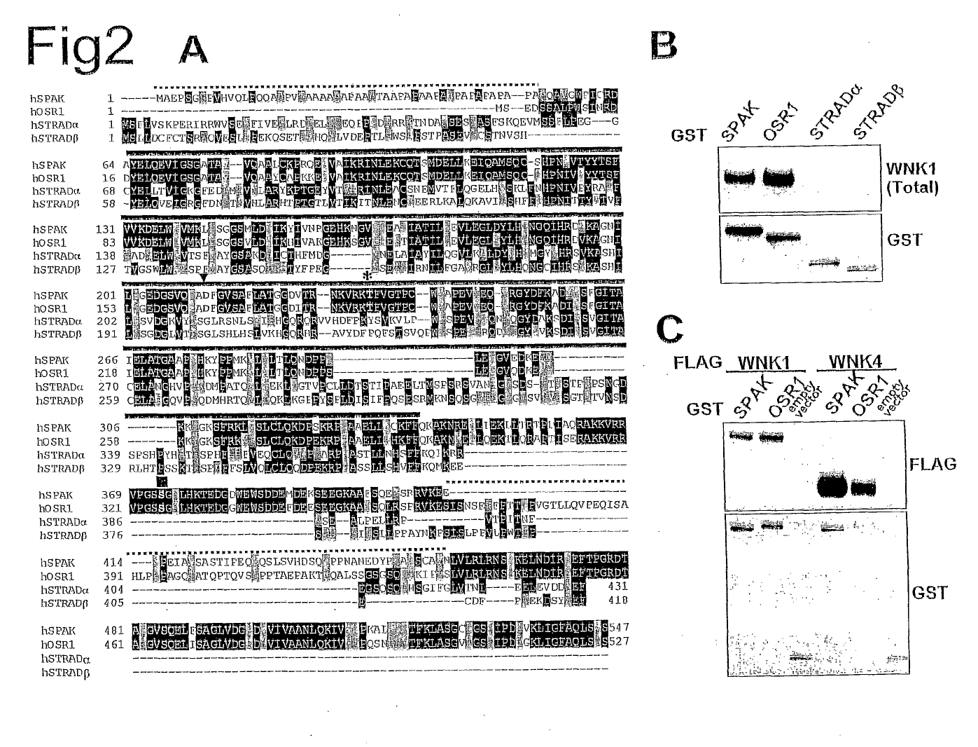

Specific interaction of WNK1 and WNK4 with SPAK and OSR1. SPAK belongs to a group of 4 highly related STE20 family kinases, the other members being OSR1 [26], STRADα and STRADβ [15, 27] (FIG. 2A). SPAK and OSR1 are reported to be active kinases and are 68% identical in sequence to one another (89% in the catalytic domain). STRADα and STRADβ are catalytically inactive pseudokinases that interact with and activate the LKB1 tumour suppressor protein kinase [15, 27] and are ˜30% identical in their pseudokinase domains to the catalytic domain of SPAK and OSR1. In order to analyse the binding of WNK1 to these STE20 family kinases, we expressed SPAK, OSR1, STRADα, and STRADβ as GST fusion proteins in 293 cells and, following affinity purification, tested whether they were associated with endogenously expressed WNK1. Both SPAK and OSR1, but neither isoform of STRAD, interacted with WNK1 (FIG. 2B). In order to determine whether WNK4 could interact with SPAK and OSR1, we overexpressed WNK4 with these kinases in 293 cells (endogenous WNK4 is not detectable in these cells), and found that WNK4 interacted with SPAK and OSR1, to a similar extent as WNK1 (FIG. 2C).

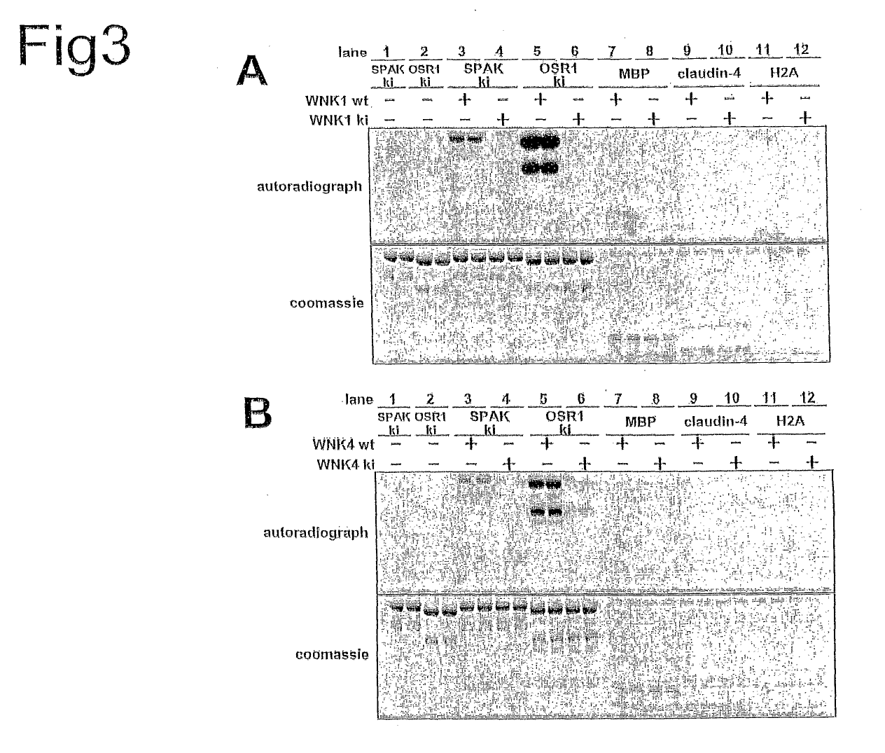

Phosphorylation of SPAK/OSR1 by WNK1/WNK4. We next tested whether an N-terminal fragment of WNK1, that encompassed residues 1-661, the largest catalytically active fragment of WNK1 that we have thus far been able to express in a soluble form [13], could phosphorylate SPAK and OSR1. We found that WNK1[1-661] expressed in E. coli (FIG. 3A) or HEK93 cells (data not shown), was capable of phosphorylating catalytically inactive mutants of SPAK[D212A] (FIG. 3A, Lane 3) or OSR1 [D164A] (FIG. 3A, Lane 5). A catalytically inactive mutant of WNK1[1-661/D368A], did not phosphorylate SPAK or OSR1, suggesting that wild type WNK1 directly phosphorylates SPAK (FIG. 3A compare lane 3 and 4) and OSR1 (FIG. 3A compare lane 5 and 6). We also observed that SPAK and OSR1 are markedly better substrates for WNK1 than either MBP, previously utilised as an in vitro substrate [1], claudin-4, previously reported to be phosphorylated by WNK4 [11], or histone 2A (H2A).

To evaluate whether WNK4 could phosphorylate SPAK and OSR1, we expressed a fragment of WNK4[1-593] in 293 cells, as we could not express WNK4 in E. coli, and found that, like WNK1, it could phosphorylate SPAK and OSR1 (FIG. 3B). A catalytically inactive mutant of WNK4[1-593/D321A] did not phosphorylate SPAK or OSR1. WNK4[1-593] did not significantly phosphorylate MBP, Claudin-4 or H2A (FIG. 3B).

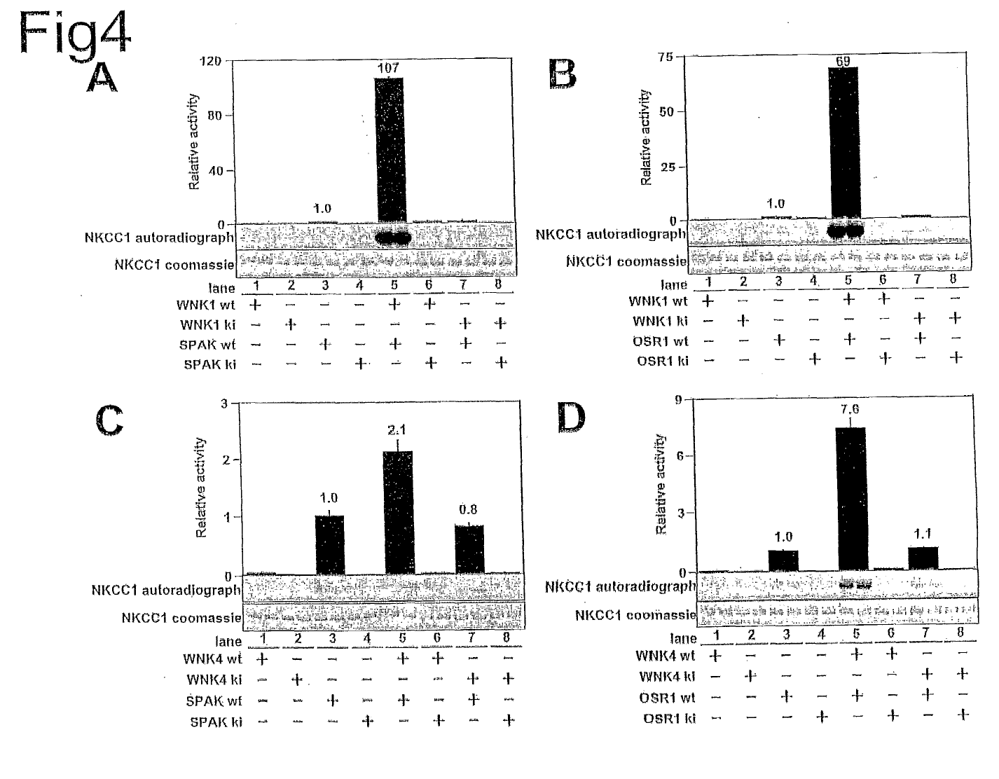

Activation of SPAK/OSR1 by WNK1/WNK4. We next tested whether WNK isoforms could activate SPAK or OSR1. In order to assess the activity of SPAK and OSR1, we employed a fragment of the Na—K—Cl cotransporter (NKCC1), encompassing the N-terminal intracellular domain (residues 1-260), that SPAK has been reported to phosphorylate [28]. Active WNK1, wild type full length SPAK or OSR1 and NKCC1[1-260] employed in these studies were expressed in E. coli to ensure that they were not contaminated with any other mammalian kinase. WNK1, SPAK or OSR1 alone did not phosphorylate NKCC1[1-260] significantly (FIGS. 4A & B lane 1 and 3). However, if SPAK or OSR1 (FIGS. 4A & B lane 5) were incubated with active WNK1[1-661], in the presence of Mg-ATP, NKCC1[1-260] was markedly phosphorylated. The extent of phosphorylation was over 100-fold higher for SPAK and over 60-fold higher for OSR1, than background levels observed with SPAK or OSR1 alone. In contrast, a catalytically inactive mutant of WNK1 failed to activate SPAK and OSR1 (FIGS. 4A & B lane 7). Moreover, catalytically inactive SPAK[D212A] or OSR1 [D164A] were not activated by WNK1[1-661] (FIGS. 4A & B lane 6). Wild type WNK4[1-593], but not catalytically inactive WNK4[1-593], was also capable of activating SPAK (FIG. 4C) and OSR1 (FIG. 4D).

Residues in SPAK and OSR1 that are phosphorylated by WNK1. To map the WNK1 phosphorylation sites in SPAK and OSR1, catalytically inactive mutants of SPAK[D212A] and OSR1[D164A] were phosphorylated by WNK1[1-661]. Under these conditions SPAK and OSR1 were phosphorylated to 0.20 and 0.36 mol of phosphate/mol, respectively. We were unable to phosphorylate the SPAK and OSR1 to a higher stoichiometry indicating that a significant proportion of the recombinant enzymes may be in a conformation that are not phosphorylated. ³²P-labelled SPAK (FIG. 5A) and OSR1 (FIG. 5C) were digested with trypsin and chromatographed on a C₁₈ column to isolate ³²P-labelled phosphopeptides. The analysis of SPAK (FIG. 5A) revealed one major phosphopeptide (P1) and three minor phosphopeptides (P2, P3 and P4). A similar profile of one major (P5) and three minor (P6 to P8) phosphopeptides was obtained for OSR1 (FIG. 5C). Mass spectrometry and solid phase Edman sequencing of the SPAK phosphopeptides established the identity of P1 and P2 as peptides phosphorylated at Ser373 and P3 and P4 as peptides, phosphorylated at Thr233 (FIG. 5A, lower panel). The OSR1 P5 and P6 peptides were phosphorylated at Ser325 (the residue equivalent to Ser373 of SPAK) and P7 and P8 peptides were phosphorylated at Thr185 (the residue equivalent to Thr233 of SPAK) (FIG. 5D). Thr233/Thr185 are located within the kinase domain T-loop, whilst Ser373/Ser325 lie in a conserved C-terminal non-catalytic region of SPAK and OSR1 (FIG. 2A).

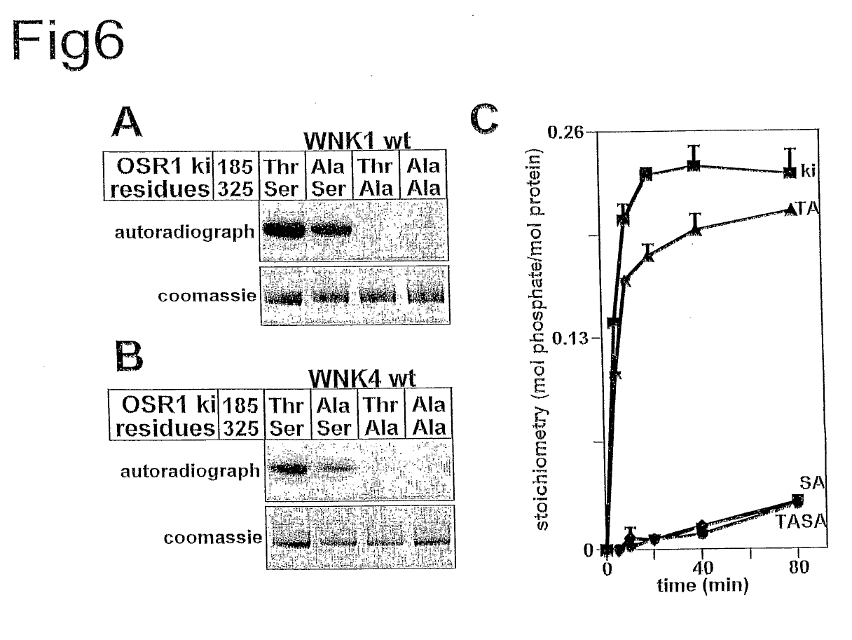

We next assessed how mutation of Thr185 and Ser325 in OSR1 affected phosphorylation by WNK1. Mutation of Thr185 moderately decreased phosphorylation of OSR1 by WNK1, whilst mutation of Ser325 virtually abolished phosphorylation of OSR1 by WNK1 (FIGS. 6A & B). Similar results were obtained employing WNK4 (FIG. 6C), indicating that WNK4 phosphorylates the same residues on OSR1 that are phosphorylated by WNK1.

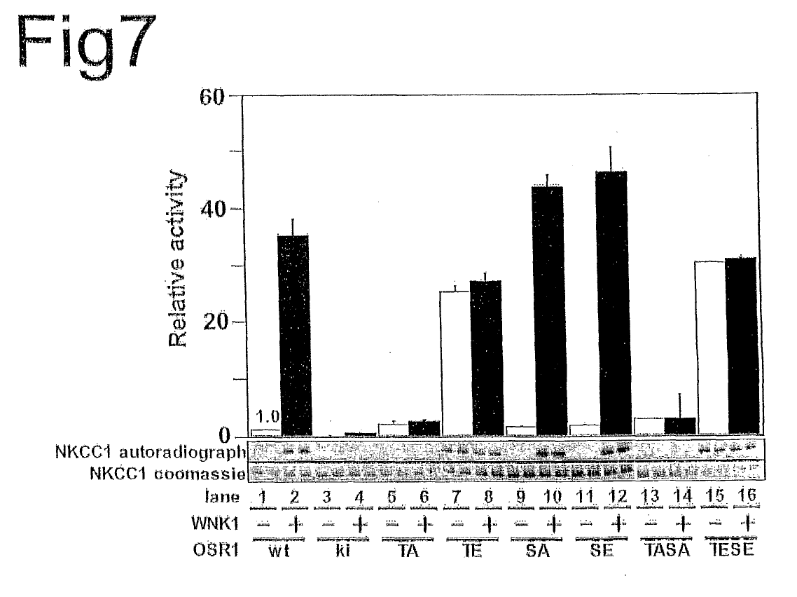

Activation of OSR1 requires Thr185 but not Ser325 phosphorylation. In order to determine the importance of the phosphorylation of Thr185 and Ser325 in regulating the activation of OSR1 by WNK1, we mutated these residues to either Ala to prevent phosphorylation, or Glu to mimic phosphorylation, and determined how this affected the activation of OSR1 by WNK1. Mutation of Thr185 to Ala, prevented WNK1 from activating OSR1 (FIG. 7, lane 6). Consistent with Thr185 phosphorylation mediating activation of OSR1, mutation of Thr185 to Glu, markedly enhanced OSR1 activity (FIG. 7 lane 7) and this mutant could not be activated further by phosphorylation with WNK1 (FIG. 7, lane 8). Mutation, of Ser325 to Ala or Glu, had no effect on the basal activity of OSR1 or on the ability of WNK1 to activate OSR1 (FIG. 7 lanes 9-12). Double OSR1 mutants in which both Thr185 and Ser325 were altered to either Ala (FIG. 7 lane 13-14) or Glu (FIG. 7 lane 15-16), had similar properties to the single Thr185 mutants.

### Discussion.

We report that endogenously expressed WNK1 in testis is associated with SPAK and that WNK1 and WNK4 can phosphorylate and activate SPAK and OSR1 in vitro. WNK isoforms are likely to have a very restricted substrate specificity as they failed to phosphorylate significantly any of ˜50 proteins or ˜500 synthetic peptides that we had previously tested (data not shown). It is a common feature of upstream kinases, such as Raf, MEK, PDK1 and LKB1, that they tend to possess highly restricted substrate specificities. Our results also indicate that activation of OSR1 by WNK1 is mediated by the phosphorylation of Thr185 within its T-loop. This is based on the finding that OSR1 is phosphorylated at Thr185 by WNK1, mutation of Thr185 to Ala abolishes activation of OSR1 by WNK and that mutation of Thr185 to Glu is sufficient to activate OSR1 in the absence of WNK1. Thus the mechanism of activation of OSR1 is similar to that of many other kinases, and is mediated by phosphorylation of the T-loop residue [29]. It is likely that SPAK will be activated by WNK1 in a similar manner to OSR1.

Interestingly, the major site that WNK1 phosphorylates in SPAK and OSR1, is not the T-loop Thr, but a Ser residue located in a C-terminal non-catalytic region of the enzyme. Both phosphorylation sites on WNK1 and WNK4, as well as the residues surrounding them, are conserved in mammalian, Xenopus, Drosophila and C. elegans homologues of SPAK/OSR1. The role that phosphorylation of the Ser residue plays is unknown, as its mutation does not affect the basal activity of OSR1, nor does it affect its ability to be activated by WNK1. Further analysis is therefore required to define the role that the Ser phosphorylation site plays in regulating SPAK and OSR1 function.

We have also observed consistently that OSR1 is phosphorylated more efficiently by WNK1 and WNK4 than SPAK (FIG. 3), despite the residues surrounding the sites of phosphorylation being almost identical in SPAK and OSR1 (FIG. 2A). Major difference between SPAK and OSR1 are the presence of a highly unusual Pro and Ala rich motif within the first 50 amino acid residues of SPAK and a region spanning ˜40 amino acids towards the C-terminal region (FIG. 2A), that might affect its folding when expressed in E. coli and/or ability to interact with WNK isoforms. SPAK has also been reported to interact with various cytoskeletal components [30], which could potentially play a scaffolding role to facilitate the phosphorylation of SPAK by WNK isoforms. We have been unable to express recombinant forms of full length non-degraded WNK1 and WNK4, due to the large size of these enzymes, and therefore it is possible that the full-length kinases would phosphorylate SPAK and OSR1, more efficiently than the catalytic fragments of WNK1 and WNK4 employed in this study.

The analysis of WNK1 and WNK4 activity has been hampered by the lack of a sensitive and quantitative in vitro assay. Previous studies relied upon measurement of the autophosphorylation activity of WNK1/WNK4 and its ability to poorly phosphorylate MBP and histone. The finding that WNK isoforms phosphorylate and activate OSR1 and SPAK, can readily be adapted to provide a sensitive and accurate method for assessing WNK isoform activity in vitro. Moreover, as WNK1^{+/-} mice possess lower blood pressure [4], and mutations in the WNK1 gene that increase its expression, lead to Gordon's hypertension syndrome in humans [3], drugs which inhibit WNK1 activity (or WNK2, WNK3 or WNK4 activity), might have utility for the treatment of this disorder and hypertension in general. The assays that we have developed in this study, could be deployed in a screen to identify small molecules inhibitors of WNK1.

SPAK was previously demonstrated to phosphorylate and activate the NKCC1 cotransporter [28]. NKKC1 was also shown to interact with SPAK and OSR1 and to localise SPAK to the apical membrane of choroids plexus epithelial cells [31]. In addition to directly activating NKCC1 by phosphorylation, SPAK was suggested to play a scaffolding role in regulating NKCC1 function, perhaps by controlling its phosphorylation by other kinases [30]. Recently, WNK4 was reported to interact with SPAK in a yeast 2 hybrid screen and evidence was presented that co-expression of WNK4 and SPAK in Xenopus oocytes increased potassium uptake through the NKCC1 transporter [32]. This study did not address whether WNK4 could phosphorylate and activate SPAK, but the finding that stimulation of NKCC1 activity was only observed following overexpression of wild type WNK4 and SPAK, but not by overexpression of inactive WNK4 or SPAK [32], is consistent with the notion that WNK4 could activate NKCC1 through its ability to phosphorylate and activate SPAK. Our findings also indicate that, in addition to WNK4 activating SPAK, WNK1 can also mediate the activation of SPAK, as well as OSR1, and could therefore play a similar role in regulating NKCC1 activity.

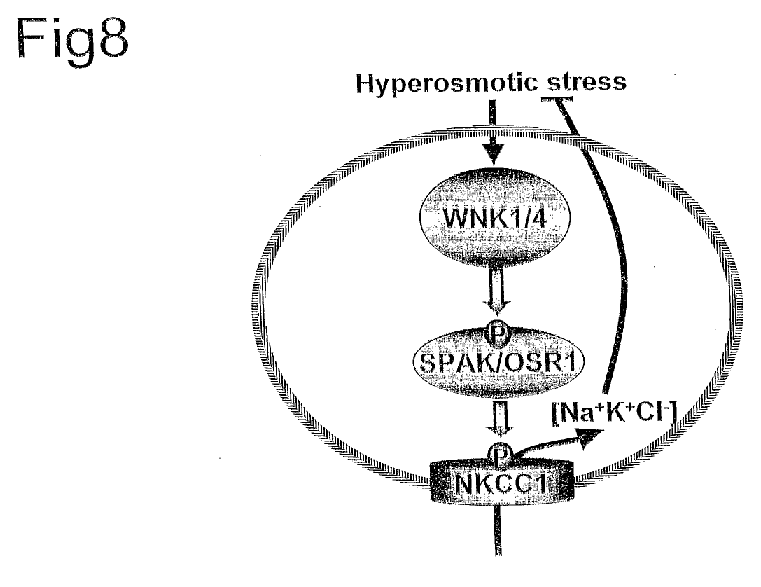

The rate of autophosphorylation of endogenously expressed WNK1 is increased ˜5-fold by hypertonic stress in kidney epithelial cells and in breast and colon cancer cell lines [33]. These conditions are well known to stimulate NKCC1 activity by inducing its phosphorylation, leading to increased uptake of Na⁺ and K⁺ ions to maintain cell volume [34, 35]. As summarised in FIG. 8, we suggest that hyperosmotic stress induces the activation of WNK isoforms through an uncharacterised mechanism, leading to the phosphorylation and activation of SPAK and OSR1, which then phosphorylate and stimulate the activity of the NKCC1 cotransporter. Significant further studies are required to validate this model. In particular, it will be important to establish that phosphorylation and activation of SPAK and/or OSR1, as well as NKCC1, is dependent on WNK activity in vivo. This may not be a trivial task due to the presence of 4 distinct isoforms of WNK in mammalian cells. It will also be important to investigate the effect of mutating the sites on SPAK/OSR1 phosphorylated by WNK1, on the ability of WNK isoforms to stimulate NKCC1 activity, and also to determine the role phosphorylation of the Ser residue phosphorylated by WNKs in controlling SPAK/OSR1 function. It would also be interesting to investigate whether any mutations in the SPAK and OSR1 protein kinases are found in humans with familial hypertension syndrome and whether SPAK and OSR1 kinases were hyperactive in subjects with Gordon's syndrome.

### EXAMPLE 2

### Assay Formats Suitable for Compound Screening

Protein kinase screening assay formats known in the art may be used, adapted in view of the identification of SPAK and OSR1 polypeptides as substrates of WNK isoform polypeptides.

For example, the techniques used in Example 1 may be used in screening compounds. Assays similar to those described in WO 03/087400 may be used. Screening assays which are capable of high throughput operation may be used. For example, assays using a substrate peptide based on one of the SPAK or OSR1 phosphorylation sites, for example using an antibody binding to the phosphorylated form of the peptide but not the unphosphorylated for (or vice versa) may be suitable.

Cell based assays may be used, for example when assessing the effect of compounds on cell volume responses.

Protein-protein binding assays may be used, for example using surface plasmon resonance-based techniques or chip-based binding assays, as well known to those skilled in the art.

SPA-based (Scintillation Proximity Assay; Amersham International) assays may be used as well known to those skilled in the art. For example, beads comprising scintillant and a substrate polypeptide, for example a SPAK or OSR1 peptide substrate as discussed above may be prepared. The beads may be mixed with a sample comprising ³²P or ³³P-γ-labelled ATP, a WNK isoform polypeptide and with the test compound. Conveniently this is done in a 96-well format. The plate is then counted using a suitable scintillation counter, using known parameters for ³²P or ³³P SPA assays. Only ³²P or ³³P that is in proximity to the scintillant, i.e. only that bound to the substrate that is bound to the beads, is detected. Variants of such an assay, for example in which the substrate polypeptide is immobilised on the scintillant beads via binding to an antibody or antibody fragment, may also be used.

### EXAMPLE 3

### Measurement of WNK1 Kinase Activity Using Peptide Substrates Derived from SPAK

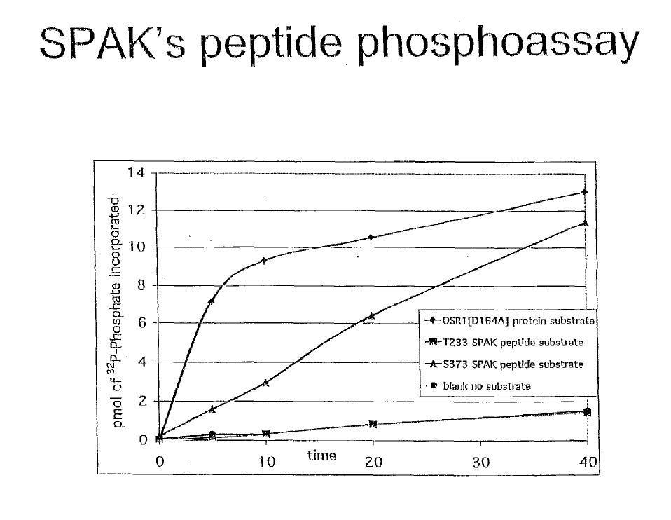

Assays were set up in a total vol of 50 μl in 50 mM Tris-HCl pH 7.5, 0.1 mM EGTA, 1 mM DTT, 10 mM MgCl₂, 0.1 mM [γ³²P]ATP (300 cpm/pmole) containing 0.1 μM of GST-WNK1(61-667), purified from E. coli, with or without the following substrates: 1 μM OSR1[D164A] or 300 μM T233 SPAK peptide (TRNKVRKTFVGTP, corresponds to residues 226 to 238 of human SPAK) or 300 μM S373SPAK peptide (vitaride RRVPGSSGHLHKT, corresponds to residues 367 to 379 of human SPAK). After incubation at 30° C. for the times indicated in FIG. 9 incorporation of ³²P-phosphate into the substrate was determined by applying the reaction mixture onto P81 phosphocellulose paper and scintillation counting after washing the papers in phosphoric acid as described previously (Alessi et al (1995)). All peptides were synthesised by Dr. Graham Bloomberg at the University of Bristol.

### EXAMPLE 4

### Assay for Inhibitors of WNK Activity

An example of a non-radioactive assay, suitable for screening of small drug-like compound libraries, has been developed using anti-phospho-SPAK antibodies to detect phosphorylation of WNK substrates SPAK or OSR1 in an ELISA format.

Anti-phospho-SPAK-S373 and anti-phospho-SPAK-T233 were anti-phospho-peptide antibodies raised in sheep for use in western blotting. They were evaluated for use in the ELISA format. Anti-phospho-SPAK-S373 was active in the ELISA with native OSR1 protein. SPAK and OSR1 are interchangeable in the assay as the amino acid sequences are very similar overall and have the same sequence flanking the phosphorylation sites. Immobilization of OSR1 to a microtitre plate, eg by absorption or capture via GST-tag, did not affect the ability of WNK1 to phosphorylate it. The catalytically inactive mutant OSR1(D164A) was chosen as the substrate of choice. Wild type OSR1 had significant autophosphorylation/ATP hydrolytic activity, and could lead to depletion of substrates during the time course of the reaction.

The assay was performed in maxisorp (Nunc) 384-clear plates. OSR1(D164A), 30 ng/well, was coated overnight at 4° C. in Tris buffered saline (TBS) pH 7.4. Excess binding sites were blocked with 5% BSA in TBS containing 0.2% Tween (TBST) for 1 hour at room temperature and then washed three times with TBST. 15 μl WNK (1-1000 ng) in reaction buffer (50 mM Tris pH 7.5, 0.01% BSA, 0.1 mM EGTA, 1 mM DTT) was added to the well and 2 μl of compound dissolved in 11% DMSO was added and incubated for 30 minutes. The reaction was initiated by the addition of 5 μL ATP (1-1000 μM)/10 mM MgCl₂ and incubated at room temperature for 25 minutes. The reaction was stopped by addition of 20 μL 0.5 M EDTA. The plates were washed three times with TBST before the addition of 22 μL anti-phospho-SPAK-S373 antibody (diluted 1:3700 fold in TBST containing 20 μg/ml blocking peptide). After 1 hour the plates were washed three times with TBST and then 22 μL of anti-sheep-peroxidase conjugate (1:5000 dilution in 1% BSA/TBST) was added to each well and incubated a further 1 hour. A final four washes of TBST were performed before addition of 22 μl peroxidase substrate 3,3′,5,5′tetramethylbenzidine TMB in 50 mM acetic acid, 50 mM sodium Acetate, 0.0009% H₂O₂. Colour was developed for 15 minutes and stopped by addition of 5 μL 1M HCl. Plates were read on an absorbance reader at 450 mm.

Alternative commercially available peroxidase substrates could be used, which would allow different colour detection. For example orthophenylenediamine (OPD) which is read at 492 nm or Diammonium-2,2′-azino-bis(3-ethyl-benzothiazoline-6-sulfonate) which is read at 405 nm. Alternative detection technologies can also be applied using fluorescent substrates such as 10-acetyl-3.7, dihydroxyphenoxazine or luminal based substrates for luminescence.

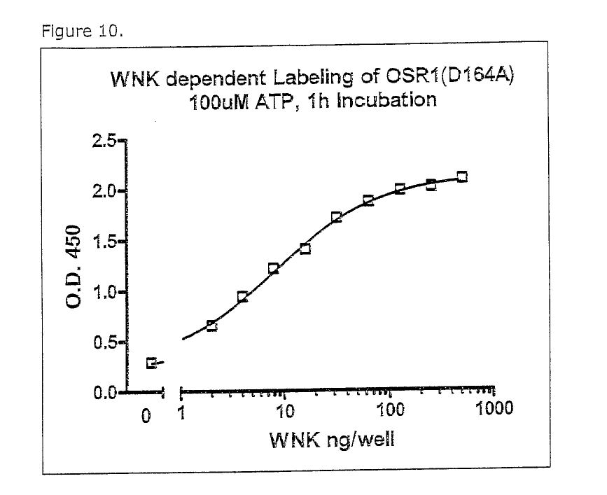

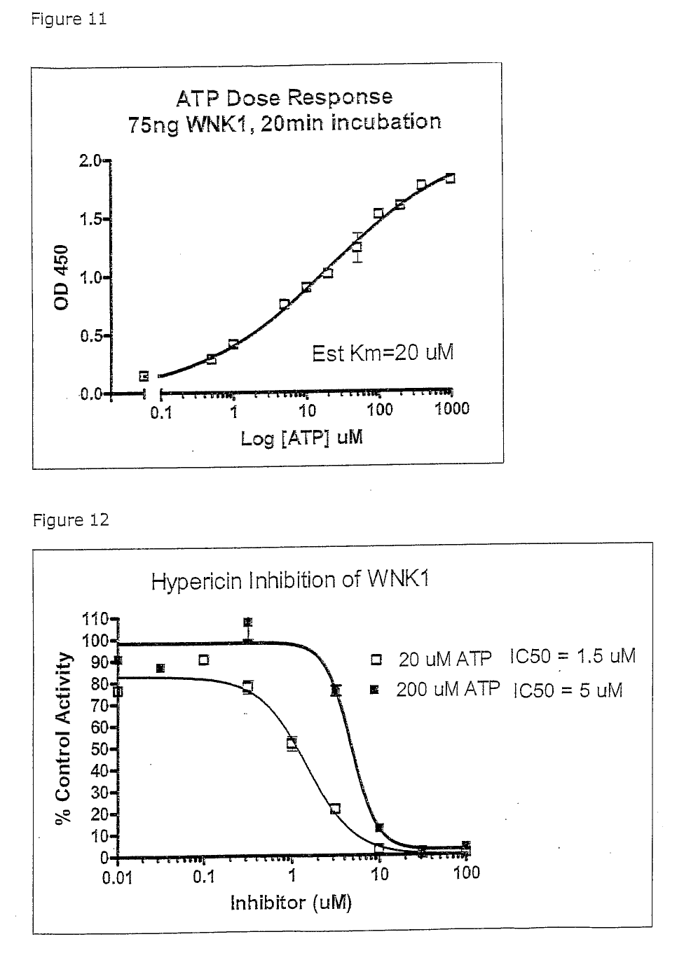

The use of the assay described to measure WNK dependent activity, an ATP dose response curve and an Inhibitor IC50 curve are shown in FIGS. 10-12. Assay statistics demonstrating a good assay window and good reproducibility obtained with this assay are shown in Table 1.

The assay was tolerant to a wide range of ATP concentrations (1-1000 μM) and 1% DMSO (compound storage solvent). Compound interference by autofluorescence, quenching or absorbance is minimised as it is heterogeneous involving several wash steps. A good assay window was obtained with Z′>0.5. Its' use to detect inhibitors was demonstrated with a small panel of broad spectrum kinase inhibitors. These included H7, ML9 and staurosporine. Hypericin was identified as an inhibitor with an IC50=1.5 μM (FIG. 3)

TABLE 1
Assay Statistics

| | OSR1 Only | WNK |
|---|---|---|
| Average | 0.199 | 1.167 |
| Standard deviation | 0.007 | 0.032 |
| Coefficient of Variation | 0.035 | 0.033 |

| | |
|---|---|
| Signal: Background | 5.85 |
| Signal: Noise | 136 |
| Z′ | 0.8 |

### REFERENCES

1 Xu, B., English, J. M., Wilsbacher, J. L., Stippec, S., Goldsmith, E. J. and Cobb, M. H. (2000) WNK1, a novel mammalian serine/threonine protein kinase lacking the catalytic lysine in subdomain II. J Biol Chem 275, 16795-16801
2 Verissimo, F. and Jordan, P. (2001) WNK kinases, a novel protein kinase subfamily in multi-cellular organisms. Oncogene 20, 5562-5569
3 Wilson, F. H., Disse-Nicodeme, S., Choate, K. A., Ishikawa, K., Nelson-Williams, C., Desitter, I., Gunel, M., Milford, D. V., Lipkin, G. W., Achard, J. M., Feely, M. P., Dussol, B., Berland, Y., Unwin, R. J., Mayan, H., Simon, D. B., Farfel, Z., Jeunemaitre, X. and Lifton, R. P. (2001) Human hypertension caused by mutations in WNK kinases. Science 293, 1107-1112
4 Zambrowicz, B. P., Abuin, A., Ramirez-Solis, R., Richter, L. J., Piggott, J., BeltrandelRIO, H., Buxton, E. C., Edwards, J., Finch, R. A., Friddle, C. J., Gupta, A., Hansen, G., Hu, Y., Huang, W., Jaing, C., Key, B. W., Jr., Kipp, P., Kohlhauff, B., Ma, Z. Q., Markesich, D., Payne, R., Potter, D. G., Qian, N., Shaw, J., Schrick, J., Shi, Z. Z., Sparks, M. J., Van Sligtenhorst, I., Vogel, P., Walle, W., Xu, N., Zhu, Q., Person, C. and Sands, A. T. (2003) Wnk1 kinase deficiency lowers blood pressure in mice: a gene-trap screen to identify potential targets for therapeutic intervention. Proc Natl Acad Sci USA 100, 14109-14114
5 Kamide, K., Takiuchi, S., Tanaka, C., Miwa, Y., Yoshii, M., Horio, T., Mannami, T., Kokubo, Y., Tomoike, H., Kawano, Y. and Miyata, T. (2004) Three novel missense mutations of WNK4, a kinase mutated in inherited hypertension, in Japanese hypertensives: implication of clinical phenotypes. Am J Hypertens 17, 446-449
6 Kahle, K. T., Wilson, F. H. and Lifton, R. P. (2005) Regulation of diverse ion transport pathways by WNK4 kinase: a novel molecular switch. Trends Endocrinol Metab 16, 98-103
7 Cope, G., Golbang, A. and O'Shaughnessy K, M. (2005) WNK kinases and the control of blood pressure. Pharmacol Ther 106, 221-231
8 Gamba, G. (2005) Role of WNK kinases in regulating tubular salt and potassium transport and in the development of hypertension. Am J Physiol Renal Physiol 288, F245-252
9 Kahle, K. T., Wilson, F. H., Leng, Q., Lalioti, M. D., O'Connell, A. D., Dong, K., Rapson, A. K., MacGregor, G. G., Giebisch, G., Hebert, S. C. and Lifton, R. P. (2003) WNK4 regulates the balance between renal NaCl reabsorption and K⁺ secretion. Nat Genet. 35, 372-376
10 Yang, C. L., Angell, J., Mitchell, R. and Ellison, D. H. (2003) WNK kinases regulate thiazide-sensitive Na—Cl cotransport. J Clin Invest 111, 1039-1045
11 Yamauchi, K., Rai, T., Kobayashi, K., Sohara, E., Suzuki, T., Itoh, T., Suda, S., Hayama, A., Sasaki, S, and Uchida, S. (2004) Disease-causing mutant WNK4 increases paracellular chloride permeability and phosphorylates claudins. Proc Natl Acad Sci USA 101, 4690-4694
12 Lee, B. H., Min, X., Heise, C. J., Xu, B. E., Chen, S., Shu, H., Luby-Phelps, K., Goldsmith, E. J. and Cobb, M. H. (2004) WNK1 phosphorylates synaptotagmin 2 and modulates its membrane binding. Mol Cell 15, 741-751
13 Vitari, A. C., Deak, M., Collins, B. J., Morrice, N., Prescott, A. R., Phelan, A., Humphreys, S, and Alessi, D. R. (2004) WNK1, the kinase mutated in an inherited high-blood-pressure syndrome, is a novel PKB (protein kinase B)/Akt substrate. Biochem J 378, 257-268
14 Sakamoto, K., McCarthy, A., Smith, D., Green, K. A., Grahame Hardie, D., Ashworth, A. and Alessi, D. R. (2005) Deficiency of LKB1 in skeletal muscle prevents AMPK activation and glucose uptake during contraction. Embo J 24, 1810-1820
15 Boudeau, J., Baas, A. F., Dealc, M., Morrice, N. A., Kieloch, A., Schutkowski, M., Prescott, A. R., Clevers, H. C. and Alessi, D. R. (2003) M025alpha/beta interact with STRADalpha/beta enhancing their ability to bind, activate and localize LKB1 in the cytoplasm. EMBO J. 22, 5102-5114
16 Harlow, E. and Lane, D. (1988) Antibodies a Laboratory Manual, Cold Spring Harbor Laboratory Publications, New York
17 Woods, Y. L., Rena, G., Morrice, N., Barthel, A., Becker, W., Guo, S., Unterman, T. G. and Cohen, P. (2001) The kinase DYRK1A phosphorylates the transcription factor FKIR at Ser329 in vitro, a novel in vivo phosphorylation site. Biochem J 355, 597-607
18 Perkins, D. N., Pappin, D. J., Creasy, D. M. and Cottrell, J. S. (1999) Probability-based protein identification by searching sequence databases using mass spectrometry data. Electrophoresis 20, 3551-3567
19 Notredame, C., Higgins, D. G. and Heringa, J. (2000) T-Coffee: A novel method for fast and accurate multiple sequence alignment. J Mol Biol 302, 205-217
20 Rice, P., Longden, I. and Bleasby, A. (2000) EMBOSS: the European Molecular Biology Open Software Suite. Trends Genet. 16, 276-277
21 Durocher, Y., Perret, S, and Kamen, A. (2002) High-level and high-throughput recombinant protein production by transient transfection of suspension-growing human 293-EBNA1 cells. Nucleic Acids Res 30, E9
22 Campbell, D. G. and Morrice, N. A. (2002) Identification of Protein Phosphorylation Sites by a Combination of Mass Spectrometry and Solid Phase Edman Sequencing. J. Biomol. Techn. 13, 121-132
23 Ushiro, H., Tsutsumi, T., Suzuld, K., Kayahara, T. and Nakano, K. (1998) Molecular cloning and characterization of a novel Step 20-related protein kinase enriched in neurons and transporting epithelia. Arch Biochem Biophys 355, 233-240
24 Scanlan, M. J., Gout, I., Gordon, C. M., Williamson, B., Stockert, E., Gure, A. O., Jager, D., Chen, Y. T., Mackay, A., O'Hare, M. J. and Old, L. J. (2001) Humoral immunity to human breast cancer: antigen definition and quantitative analysis of mRNA expression. Cancer Immun 1, 4
25 Yang, C. L., Zhu, X., Wang, Z., Subramanya, A. R. and Ellison, D. H. (2005) Mechanisms of WNK1 and WNK4 interaction in the regulation of thiazide-sensitive NaCl cotransport. J Clin Invest 115, 1379-1387
26 Tamari, M., Daigo, Y. and Nakamura, Y. (1999) Isolation and characterization of a novel serine threonine kinase gene on chromosome 3p22-21.3. J Hum Genet. 44, 116-120
27 Baas, A. F., Boudeau, J., Sapkota, G. P., Smit, L., Medema, R., Morrice, N. A., Alessi, D. R. and Clevers, H. C. (2003) Activation of the tumour suppressor kinase LKB1 by the STE20-like pseudokinase STRAD. EMBO J. 22, 3062-3072
28 Dowd, B. F. and Forbush, B. (2003) PASK (proline-alanine-rich STE20-related kinase), a regulatory kinase of the Na—K—Cl cotransporter (NKCC1). J Biol Chem 278, 27347-27353
29 Nolen, B., Taylor, S, and Ghosh, G. (2004) Regulation of protein kinases; controlling activity through activation segment conformation. Mol Cell 15, 661-675
30 Piechotta, K., Garbarini, N., England, R. and Delpire, E. (2003) Characterization of the interaction of the stress kinase SPAK with the Na⁺—K⁺-2Cl— cotransporter in the nervous system: evidence for a scaffolding role of the kinase. J Biol Chem 278, 52848-52856
31 Piechotta, K., Lu, J. and Delpire, E. (2002) Cation chloride cotransporters interact with the stress-related kinases Step 20-related proline-alanine-rich kinase (SPAK) and oxidative stress response 1 (OSR1). J Biol Chem 277, 50812-50819
32 Gagnon, K. B., England, R. and Delpire, E. (2005) Volume sensitivity of cation-chloride cotransporters is modulated by the interaction of two kinases: SPAK and WNK4. Am J Physiol Cell Physiol
33 Lenertz, L. Y., Lee, B. H., Min, X., Xu, B. E., Wedin, K., Earnest, S., Goldsmith, E. J. and Cobb, M. H. (2005) Properties of WNK1 and implications for other family members. J Biol Chem
34 Kurihara, K., Moore-Hoon, M. L., Saitoh, M. and Turner, R. J. (1999) Characterization of a phosphorylation event resulting in upregulation of the salivary Na(+)—K(+)-2Cl(−) cotransporter. Am J Physiol 277, C1184-1193
35 Lytle, C. and Forbush, B., 3rd. (1992) The Na—K—Cl cotransport protein of shark rectal gland. II. Regulation by direct phosphorylation. J Biol Chem 267, 25438-25443

1. A method for identifying a compound expected to be useful in modulating a WNK (With No Lysine Kinase) isoform protein kinase activity, the method comprising the steps of (1) determining whether a test compound modulates the protein kinase activity of a WNK isoform polypeptide on a substrate SPAK (STE20/SPS1-related Proline-Alanine-rich Kinase) polypeptide or OSR1 (Oxidative Stress Response kinase-1) polypeptide and (2) selecting a compound which modulates the said WNK isoform polypeptide protein kinase activity.

2. A method for identifying a compound expected to be useful in modulating, for example inhibiting, the phosphorylation of SPAK or OSR1 in a cell, the method comprising the steps of (1) determining whether a test compound modulates, for example inhibits, the protein kinase activity of a WNK isoform polypeptide, and (2) selecting a compound which modulates, for example inhibits, the protein kinase activity of the WNK isoform polypeptide.

3. A method of identifying a compound expected to be useful in modulating, for example inhibiting, the phosphorylation of SPAK or OSR1 in a cell, comprising the step of determining the effect of the compound on the ability of a WNK isoform polypeptide to bind to a SPAK polypeptide or OSR1 polypeptide and (2) selecting a compound which modulates, for example inhibits, the ability of said WNK isoform polypeptide to bind to the SPAK or OSR1 polypeptide.

4. The method of claim 1, 2 or 3 wherein the WNK isoform polypeptide is a WNK1 or WNK4 polypeptide.

5. The method of claim 1, 2 or 3 wherein the WNK isoform polypeptide and/or SPAK or OSR1 polypeptide is recombinant.

6. A mutated SPAK polypeptide wherein the residue corresponding to Ser373 is mutated, for example to an alanine residue or to an aspartate or glutamate residue; and/or wherein the residue corresponding to D212 is mutated, for example to an alanine residue.

7. A mutated OSR1 polypeptide wherein the T-loop threonine residue corresponding to Thr185 of human OSR1 is mutated, for example to an alanine residue or to an aspartate or glutamate residue; and/or wherein the residue corresponding to Ser325 is mutated, for example to an alanine residue or to an aspartate or glutamate residue; and/or wherein the residue corresponding to D164 is mutated, for example to an alanine residue.

8. A polynucleotide encoding a mutated SPAK or OSR1 polypeptide according to claim 6 or 7.

9. A method of activating a SPAK polypeptide or OSR1 polypeptide wherein the SPAK polypeptide or OSR1 polypeptide is phosphorylated by a WNK isoform polypeptide.

10. Use of a WNK isoform polypeptide in a method of phosphorylating a SPAK polypeptide or OSR1 polypeptide.

11. The method of claim 9 wherein the OSR1 polypeptide is phosphorylated on the T-loop threonine residue corresponding to Thr185 of full length human OSR1; or wherein the SPAK polypeptide is phosphorylated on the T-loop threonine residue corresponding to Thr233 of full length human SPAK.

12. The method according to claim 9 or claim 10 wherein the OSR1 polypeptide is further phosphorylated on the residue equivalent to Ser325 of full-length human OSR1; or wherein the SPAK polypeptide is further phosphorylated on the residue equivalent to Ser373 of full length human SPAK.

13. The method of any one of claims 1 to 3 comprising the step of assessing whether the compound modulates NKCC1 activity in a whole cell, tissue or organism; or blood pressure in an organism and a compound that modulates the activity or blood pressure is selected.

14. The method of claim 13 further comprising the step of assessing whether the compound modulates the activity of a WNK isoform and/or SPAK or OSR1 in the whole cell, tissue or organism, and a compound that modulates the activity is selected.

15. The method of any one of claims 1 to 3 further comprising the step of synthesising, purifying and/or formulating the selected compound.

16. A method for preparing a compound which modulates the activity of a WNK isoform or SPAK or OSR1, the method comprising 1) performing a method according to any one of claims 1 to 3 and 2) synthesising, purifying and/or formulating the selected compound.

17. A specific binding partner, typically an antibody, that binds in a phosphorylation state-sensitive manner to an epitope encompassing Ser373 of SPAK (for example human SPAK), or Thr185 or Ser 325 of OSR1 (for example human OSR1).

18. A peptide comprising a fragment derivable from SPAK which encompasses the Ser373 residue of SPAK and at least part of the sequence which includes this residue, for example at least the 2, 3, 4, 5, 6 or 7 residues C-terminal or N-terminal of this residue, wherein the fragment has less than 50, 40, 30, 25, 20, 15 or 10 residues.

19. A peptide comprising a fragment derivable from OSR1 which encompasses the Ser325 residue of OSR1 or the Thr185 residue of OSR1 and at least part of the sequence which includes this residue, for example at least the 2, 3, 4, 5, 6 or 7 residues C-terminal or N-terminal of this residue, wherein the fragment has less than 50, 40, 30, 25, 20, 15 or 10 residues.
